(19) United States
(12) Patent Application Publication
(10) Pub. No.: US 2021/0274789 A1
(43) Pub. Date: Sep. 9, 2021

(54) Antimicrobial Surface Treatment

(71) Applicant: Kode Biotech Limited, Auckland (NZ)

(72) Inventors: Stephen Michael Henry, Auckland (NZ); Pavithra Ranguraman, Auckland (NZ)

(21) Appl. No.: 17/255,198

(22) PCT Filed: Jun. 24, 2019
(86) PCT No.: PCT/IB2019/055305

§ 371 Date: Dec. 22, 2020

(30) Foreign Application Priority Data
Jun. 22, 2018 (AU) 2018902248
Sep. 7, 2018 (AU) 2018903364

(51) Int. Cl. A01N 59/16; A01N 33/12; A01N 25/10; A61L 26/00; A61L 27/34; A61L 27/54

(52) U.S. Cl. CPC A01N 59/16; A01N 33/12; A01N 25/10; A61L 26/0028; A61L 27/34; A61L 2300/102; A61L 27/54; A61L 2430/12; A61L 2300/404; A61L 2420/02; A61L 26/0066

(57) ABSTRACT

A method of treating a surface so that the antimicrobial activity of water-soluble antimicrobial agents is retained at the surface despite repeated washing with water is described. The method uses a dispersion of a lipidated polyanionic molecule and one or more antimicrobial agents in an aqueous carrier such as water. The treatment of the surface of the fabric of adhesive bandages and stainless steel is demonstrated using a combination of crystal violet and silver nitrate as the antimicrobial agents. The method has application in the treatment of such surfaces to control microbial growth and the development of biofilms.

### CROSS REFERENCE TO RELATED APPLICATIONS

This application is a 371 national stage filing of international patent application having serial no. PCT/IB2019/055305, filed Jun. 24, 2019, which claims priority to, and the benefit of, AU provisional applications having serial no. 2018903364, filed Sep. 7, 2018, and serial no. 2018902248, filed Jun. 22, 2018, all of which are hereby incorporated by reference in their entireties.

### TECHNICAL FIELD

The invention relates to antimicrobial surface treatments. In particular, the invention relates to a method of localising and retaining the antimicrobial activity of water-soluble antimicrobial agents at the surface of a substrate such as the fabric of an adhesive bandage or the ceramic or metal of a dental or medical implant or prosthesis.

### BACKGROUND ART

The publications of Adams (1967) and Docampo and Moreno (1990) disclose the antibacterial action of crystal violet (also known as gentian violet). Although still used as a histological stain and in Gram's method of classifying bacteria, the medical use of this dye has been largely superseded by more modern drugs. The publication of Bovin et al (2016) discloses the use of a lipid conjugated functional moiety to impart an antimicrobial activity to a surface. In this publication the functional moiety is a selenide, such as cyanoselenide. The functional moiety is covalently linked to a diacylglycerophospholipid, such as phosphatidylethanolamine, via a rigid polycarboxylated spacer comprising repeat carboxymethylglycine (CMG) units.

It is an object of the present invention to provide a method of augmenting the antimicrobial activity imparted to the surface of a substrate by the use of the conjugates disclosed in the publication of Bovin et al (2016). It is also an object of the present invention to provide a method of using other lipidated polyanionic molecules to impart the antimicrobial activity of one or more antimicrobial agents to a surface without the requirement for the antimicrobial agents to be covalently linked to a lipid. These objects are to be read in the alternative with the object at least to provide a useful choice in the selection of methods of imparting an antimicrobial activity to a surface.

### SUMMARY OF INVENTION

In a first aspect the invention provides a method of treating a surface to retain the antimicrobial activity of one or more water-dispersible antimicrobial agents at the surface when the surface is contacted with an aqueous vehicle, comprising the step of contacting the surface with a lipidated polyanionic molecule dispersed in a solution of the one or more water-dispersible antimicrobial agents. Typically, the contacted surface is then dried.

Preferably, the one or more water-dispersible antimicrobial agents are selected from the group consisting of: silver and salts of hexamethylpararosaniline. The silver may be in its elemental form, e.g. silver nanoparticles, or in the form of a water-soluble salt, e.g. silver nitrate (AgNO₃). Preferably, the silver is in the form of silver nitrate (AgNO₃). Preferably, the salt of hexamethylpararosaniline is hexamethylpararosaniline chloride (crystal violet).

Preferably, the one or more water-dispersible antimicrobial agents are water-soluble. More preferably, the one or more water-dispersible antimicrobial agents consist of: water-soluble salts of silver and water-soluble salts of hexamethylpararosaniline. More preferably, the one or more water-dispersible antimicrobial agents consist of silver nitrate (AgNO₃) and hexamethylpararosaniline chloride (crystal violet).

Preferably, the surface is selected from the group consisting of ceramics, metals and polymers. Metals include gold, silver and stainless steel. Polymers include cellulose ethyl sulphonate. Preferably, the surface is selected from the group consisting of: the fabric of an adhesive bandage and the stainless steel of a dental or medical implant.

Preferably, the surface is not charged.

Preferably, the aqueous vehicle is plasma.

The lipidated polyanionic molecule is of the structure L-A-M where L is a lipid, A is an optional linker covalently linking L to M, and M is the polyanionic molecule. The polyanionic molecule is a short chain linear polymer, i.e. an oligomer, comprising 2 to 8 repeated units. Preferably, the polyanionic molecule is a polyacid. More preferably, the polyanionic molecule is a polyacid selected from the group consisting of polycarboxylic, polyphosphoric and polysulfonic acids. Most preferably, the polyanionic molecule is a polycarboxylic acid.

The polyanionic molecule is a weak acid meaning that the fractional charge of the molecule is dependent on factors such as the pH of the solution, counterion concentration or ionic strength. Examples of weak polyanionic molecules are the polyacrylic acid and poly(4-ethenylbenzenesulfonic acid).

Preferably, L is a lipid selected from the group consisting of: monoacyl-, monoalkyl-, diacyl- and dialkyl-lipids. More preferably, L is a lipid selected from the group consisting of: diacyl- and dialkyl-lipids. Yet more preferably, L is a lipid selected from the group consisting of: diacyl- and dialkyl-glycerolipids. Yet even more preferably, L is a lipid selected from the group consisting of: diacyl- and dialkyl-glycerphospholipids. Most preferably, L is the lipid phosphatidylalkolamine of the structure:

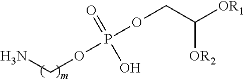

where m is the integer 1, 2 or 3 and R₁ and R₂ are independently selected from the group consisting of: saturated, mono-unsaturated and di-unsaturated C₁₂₋₄₄-acyl and saturated, mono-unsaturated and di-unsaturated C₁₄₋₂₂-alkyl substituents.

Preferably, R₁ and R₂ are independently selected from the group consisting of: saturated and mono-unsaturated C₁₆₋₂₀-acyl substituents. More preferably, R₁ and R₂ are independently selected from the group consisting of: oleoyl and stearyl substituents. Yet more preferably, m is the integer 2 and L is the lipid phosphatidylethanolamine. Most preferably, R₁ and R₂ are oleoyl substituents and L is the lipid dioleoylphosphatidylethanolamine (DOPE).

Preferably, the lipidated polyanionic molecule is a lipidated polycarboxylic molecule. More preferably, the lipidated polyanionic molecule is a lipidated polycarboxylic molecule of the structure:

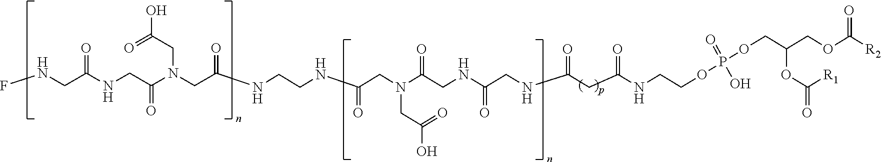

where F is H or a functional moiety, n is the integer 1, 2 or 4, p is the integer 3, 4 or 5. Most preferably, F is H, n is 2 and p is 4.

The aqueous vehicle may be water, saline or plasma.

Preferably, when F is a functional moiety it is selenocyanate.

Preferably, the solution is in saline or water. More preferably, the solution is in water.

Preferably, the antimicrobial activity is an antibacterial activity. More preferably, the antimicrobial activity is an antibacterial activity against bacteria of one or more species selected from the group consisting of: Staphylococcus aureus, Staphylococcus epidermidis, Pseudomonas aeruginosa and Escherichia coli.

In a second aspect the invention provides a composition for use in the first aspect of the invention consisting essentially of a water-soluble salt of hexamethylpararosaniline, a water-soluble salt of silver, a lipidated polyanionic molecule and water.

In a third aspect the invention provides a fabric having a surface treated in accordance with the method of the first aspect of the invention.

In a fourth aspect the invention provides a surgical implant having a surface treated in accordance with the method of the first aspect of the invention.

In the description and claims of this specification the following abbreviations, acronyms, terms and phrases have the meaning provided: “acyl” means a radical of general formula —C(O)R, where R is an alkyl group, derived from a carboxylic acid; “alkyl” means a hydrocarbon radical derived from an alkane by removal of a hydrogen atom; “aqueous vehicle” means a solvent containing water; “CAS RN” means Chemical Abstracts Service (CAS, Columbus, Ohio) Registry Number; “cfu” means colony forming unit; “comprising” means “including”, “containing” or “characterized by” and does not exclude any additional element, ingredient or step; “consisting essentially of” means excluding any element, ingredient or step that is a material limitation; “consisting of” means excluding any element, ingredient or step not specified except for impurities and other incidentals; “crystal violet” means N-[4-[bis[4-(dimethylamino)phenyl]methylene]-2,5-cyclohexadien-1-ylidene]-N-methyl-methanaminium chloride [CAS RN 548-62-9]; “lipidated” means conjugated to a lipid; “plasma” means the colourless fluid part of blood or lymph; “polyanionic” means having multiple negative electric charges; “polycarboxylic” means having multiple carboxylate groups; and “water-soluble” means having a solubility in water of at least 50 g/L at 27° C. A paronym of any of these defined terms has a corresponding meaning.

The terms “first”, “second”, “third”, etc. used with reference to elements, features or integers of the subject matter defined in the Statement of Invention and Claims, or when used with reference to alternative embodiments of the invention are not intended to imply an order of preference.

Where concentrations or ratios of reagents are specified the concentration or ratio specified is the initial concentration or ratio of the reagents. Where values are expressed to one or more decimal places standard rounding applies. For example, 1.7 encompasses the range 1.650 recurring to 1.749 recurring.

Where reference is made to the charge of a chemical group or species reference is being made to the charge of that group or species at pH 7.4.

In the representations of chemical structures, and in the absence of further limitation, the use of plain bonds in the representations of the structures of compounds encompasses the diastereomers, enantiomers and mixtures thereof of the compounds. Parentheses and brackets, ( )ₘ and [ ]ₙ, are used to denote a repeated chemical group where the group contained in the brackets or parentheses is repeated m or n times, respectively.

The invention will now be described with reference to embodiments or examples and the figures of the accompanying drawings pages.

### BRIEF DESCRIPTION OF DRAWINGS

FIG. 1. A template (A) for the 24-well cell culture plate (B) and scanning electron micrographs obtained for the swatches incubated in well A1 (C, upper micrograph) and well A2 (C, lower micrograph). The growth is consistent with the colour change from dark blue (no growth) in well A1 to light red (growth) in well A2.

FIG. 2. A template (A) for the wells of a 24-well plate (B) in which rows 2 to 6 correspond to decreasing concentrations of construct, columns A and B are duplicate unwashed treated substrates and columns C and D are duplicate washed treated substrates. Row 1 corresponds to the controls, i.e. unwashed and washed untreated substrates. The decreased retention of crystal violet with decreasing concentration of construct in both unwashed and washed treated substrates is indicated by the lightening of the colour of the substrate when descending from row 2 to row 6.

FIG. 3. Absorbances measured for washings of the treated substrates with 95% (v/v) ethanol. Increasing absorbances, i.e. increasing quantities of retained crystal violet, were measured for substrates treated with increasing concentrations of construct.

FIG. 4. Growth (log cfu) in Mueller Hinton Broth of an inoculum of 2.7×10⁶ cells of an isolate of Staphylococcus aureus in the presence of a swatch (0.25 cm²) of the fabric from a BAND-AID™ adhesive bandage (Johnson & Johnson). Swatch (BAND-AID™) contacted with a dispersion in water of the lipidated polyanionic molecule designated FSL-biotin, crystal violet and silver nitrate (FSL-BCA). Swatch (BAND-AID™) contacted with a solution in water of crystal violet and silver nitrate (CVA@1.6 mM). Swatch (0.25 cm²) of fabric from a commercially available silver bandage (DURAFIBRE™ AG, Smith and Nephew) (Ag bandage). Untreated swatch (BAND-AID™) with inoculum (positive growth control) (PC). Untreated swatch (BAND-AID™) with no inoculum (negative growth control) (NC).

FIG. 5. Scanning electron micrographs (×500) of the surface of treated (FSL-0, crystal violet and silver nitrate)(A) and untreated (B) swatches of the fabric from a BAND-AID™ adhesive bandage (Johnson & Johnson) following incubation in Mueller Hinton Broth in the presence of an inoculum of 8×10⁶ cells of an isolate of Staphylococcus aureus.

FIG. 6. Growth (log cfu) in Mueller Hinton Broth of an inoculum of 8×10⁶ cells of an isolate of Staphylococcus aureus in the presence of treated (FSL-biotin, crystal violet and silver nitrate)(FSL-BCA) or untreated (PC) swatches (0.25 cm²) of the fabric from a BAND-AID™ adhesive bandage (Johnson & Johnson).

FIG. 7. A template (A) for the wells of a 24-well plate (B) showing no growth (dark blue) in rows 1 and 2 and growth (light red) in row 3. Negative controls (clear; no resazurin) are provided in row 4. Wells in row 1 contained stamped coupons of stainless steel (SS316) that had been contacted with a dispersion in water of the lipidated polyanionic molecule designated FSL-biotin, crystal violet and silver nitrate. Wells in row 2 contained stamped coupons of stainless steel (SS316) that had been contacted with a dispersion in water of the lipidated polyanionic molecule designated FSL-0, crystal violet and silver nitrate. Wells in row 3 contained untreated stamped coupons of stainless steel (SS316). Wells in columns A and B were inoculated with 8.3×10⁶ cells and wells in columns C and D were inoculated with 8.3×10⁵ cells of an isolate of Staphylococcus aureus.

FIG. 8. Duplicate wells from a 24-well plate showing no growth (dark blue) and growth (light red) in wells containing stamped stainless steel (SS316) wells that had been either contacted with a dispersion in water of the lipidated polyanionic molecule designated FSL-biotin, crystal violet and silver nitrate (A) or untreated (B). An inoculum of 8×10⁴ cells of an isolate of Staphylococcus aureus was added to each well.

FIG. 9. Scanning electron micrographs (×2,000) of the surfaces of the stamped stainless steel wells recovered from the wells of the 24-well plate shown in FIG. 8A(A) and FIG. 8B(B).

FIG. 10. Growth (log cfu) in Mueller Hinton Broth of an inoculum of 2.13×10⁶ cells of an isolate of Staphylococcus epidermis in the presence of a swatch (0.25 cm²) of the fabric from a BAND-AID™ adhesive bandage (Johnson & Johnson). Swatch (BAND-AID™) contacted with a dispersion in water of the lipidated polyanionic molecule designated FSL-biotin, crystal violet and silver nitrate (FSL-BCA). Swatch (BAND-AID™) contacted with a solution in water of crystal violet and silver nitrate (CVA@1.6 mM). Swatch (0.25 cm²) of fabric from a commercially available silver bandage (DURAFIBRE™ AG, Smith and Nephew) (Ag bandage). Untreated swatch (BAND-AID™) with inoculum (positive growth control) (PC). Untreated swatch (BAND-AID™) with no inoculum (negative growth control) (NC).

FIG. 11. Scanning electron micrographs (×500) of the surface of treated (FSL-biotin, crystal violet and silver nitrate) (FSL-BCA) and untreated (PC) swatches of the fabric from a BAND-AID™ adhesive bandage (Johnson & Johnson) following incubation in Mueller Hinton Broth in the presence of an inoculum of 10⁷ cells of an isolate of Staphylococcus epidermis.

FIG. 12. Growth (log cfu) in Mueller Hinton Broth of an inoculum of 10⁷ cells of an isolate of Staphylococcus epidermis in the presence of treated (FSL-biotin, crystal violet and silver nitrate) (FSL BCA) or untreated (PC) swatches (0.25 cm²) of the fabric from a BAND-AID™ adhesive bandage (Johnson & Johnson).

FIG. 13. A template (A) for the wells of a 24-well plate (B) showing no growth (dark blue) in rows 1 and 2 and in wells C3 and D3 and growth (light red) in wells A3 and B3. Negative controls (clear; no resazurin) are provided in row 4. Wells in row 1 contained stamped coupons of stainless steel (SS316) that had been contacted with a dispersion in water of the lipidated polyanionic molecule designated FSL-biotin, crystal violet and silver nitrate. Wells in row 2 contained stamped coupons of stainless steel (SS316) that had been contacted with a dispersion in water of the lipidated polyanionic molecule designated FSL-0, crystal violet and silver nitrate. Wells in row 3 contained untreated stamped coupons of stainless steel (SS316). Wells in columns A and B were inoculated with 8.3×10⁶ cells and wells in columns C and D were inoculated with 8.3×10⁵ cells of an isolate of Staphylococcus epidermis.

FIG. 14. Growth (log cfu) in Mueller Hinton Broth of an inoculum of 1.51×10⁶ cells of an isolate of Pseudomonas aeruginosa in the presence of a swatch (0.25 cm²) of the fabric from a BAND-AID™ adhesive bandage (Johnson & Johnson). Swatch (BAND-AID™) contacted with a dispersion in water of the lipidated polyanionic molecule designated FSL-biotin, crystal violet and silver nitrate (FSL-BCA). Swatch (BAND-AID™) contacted with a solution in water of crystal violet and silver nitrate (CVA@1.6 mM). Swatch (0.25 cm²) of fabric from a commercially available silver bandage (DURAFIBRE™ AG, Smith and Nephew) (Ag bandage). Untreated swatch (BAND-AID™) with inoculum (positive growth control) (PC). Untreated swatch (BAND-AID™) with no inoculum (negative growth control) (NC).

FIG. 15. A scanning electron micrograph (×1,000) of the surface of treated (FSL-biotin, crystal violet and silver nitrate) swatch (A) and a scanning electron micrograph (×2,000) of the surface of an untreated swatch (B) of the fabric from a BAND-AID™ adhesive bandage (Johnson & Johnson) following incubation in Mueller Hinton Broth in the presence of an inoculum of 8.7×10⁶ cells of an isolate of Pseudomonas aeruginosa.

FIG. 16. Growth (log cfu) in Mueller Hinton Broth of an inoculum of 8.7×10⁶ cells of an isolate of Pseudomonas aeruginosa in the presence of treated (FSL-biotin, crystal violet and silver nitrate) (FSL-BCA) or untreated (PC) swatches (0.25 cm²) of the fabric from a BAND-AID™ adhesive bandage (Johnson & Johnson).

FIG. 17. A template (A) for the wells of a 24-well plate (B) showing no growth (dark blue) in rows 1 and 2 and growth (light red) in row 3. Negative controls (clear; no resazurin) are provided in row 4. Wells in row 1 contained stamped coupons of stainless steel (SS316) that had been contacted with a dispersion in water of the lipidated polyanionic molecule designated FSL-biotin, crystal violet and silver nitrate. Wells in row 2 contained stamped coupons of stainless steel (SS316) that had been contacted with a dispersion in water of the lipidated polyanionic molecule designated FSL-0, crystal violet and silver nitrate. Wells in row 3 contained untreated stamped coupons of stainless steel (SS316). Wells in columns A and B were inoculated with 2.1×10⁶ cells and wells in columns C and D were inoculated with 2.1×10⁵ cells of an isolate of Pseudomonas aeroginosa.

FIG. 18. Duplicate wells from a 24-well plate showing no growth (dark blue) and growth (light red) in wells containing stamped stainless steel (SS316) wells that had been either contacted with a dispersion in water of the lipidated polyanionic molecule designated FSL-biotin, crystal violet and silver nitrate (A) or untreated (B). An inoculum of 2.1×10⁶ cells of an isolate of Pseudomonas aeruginosa was added to each well.

FIG. 19. Scanning electron micrographs (×2,000) of the surfaces of the stamped stainless steel wells recovered from the wells of the 24-well plate shown in FIG. 18A(A) and FIG. 18B(B).

FIG. 20. Growth (log cfu) in Mueller Hinton Broth of an inoculum of 1.2×10⁶ cells of an isolate of Escherichia coli in the presence of a swatch (0.25 cm²) of the fabric from a BAND-AID™ adhesive bandage (Johnson & Johnson). Swatch (BAND-AID™) contacted with a dispersion in water of the lipidated polyanionic molecule designated FSL-biotin, crystal violet and silver nitrate (FSL-BCA-Ag). Swatch (BAND-AID™) contacted with a solution in water of crystal violet and silver nitrate (CVA@1.6 mM). Swatch (0.25 cm²) of fabric from a commercially available silver bandage (DURAFIBRE™ AG, Smith and Nephew) (Ag bandage). Untreated swatch (BAND-AID™) with inoculum (positive growth control) (PC). Untreated swatch (BAND-AID™) with no inoculum (negative growth control) (NC).

FIG. 21. Scanning electron micrographs (×1,000) of the surface of treated (FSL-biotin, crystal violet and silver nitrate) (FSL-BCA) and untreated (PC) swatches of the fabric from a BAND-AID™ adhesive bandage (Johnson & Johnson) following incubation in Mueller Hinton Broth in the presence of an inoculum of 3.7×10⁶ cells of an isolate of Escherichia coli.

FIG. 22. Growth (log cfu) in Mueller Hinton Broth of an inoculum of 3.7×10⁶ cells of an isolate of Escherichia coli in the presence of treated (FSL-biotin, crystal violet and silver nitrate)(FSL-BCA) or untreated (PC) swatches (0.25 cm²) of the fabric from a BAND-AID™ adhesive bandage (Johnson & Johnson).

FIG. 23. A template (A) for the wells of a 24-well plate (B) showing no growth (dark blue) in rows 1 and 2 and growth (light red) in row 3. Negative controls (clear; no resazurin) are provided in row 4. Wells in row 1 contained stamped coupons of stainless steel (SS316) that had been contacted with a dispersion in water of the lipidated polyanionic molecule designated FSL-biotin, crystal violet and silver nitrate. Wells in row 2 contained stamped coupons of stainless steel (SS316) that had been contacted with a dispersion in water of the lipidated polyanionic molecule designated FSL-0, crystal violet and silver nitrate. Wells in row 3 contained untreated stamped coupons of stainless steel (SS316). Wells in columns A and B were inoculated with 2.8×10⁶ cells and wells in columns C and D were inoculated with 2.8×10³ cells of an isolate of Escherichia coli.

FIG. 24. Duplicate wells from a 24-well plate showing no growth (dark blue) and growth (light red) in wells containing stamped stainless steel (SS316) wells that had been either contacted with a dispersion in water of the lipidated polyanionic molecule designated FSL-biotin, crystal violet and silver nitrate (A) or untreated (B). An inoculum of 1.3×10⁵ cells of an isolate of Escherichia coli was added to each well.

FIG. 25. Scanning electron micrographs (×2,000) of the surfaces of the stamped stainless steel wells recovered from the wells of the 24-well plate shown in FIG. 24A(A) and FIG. 24B(B).

FIG. 26. Growth (log cfu) in Mueller Hinton Broth of an inoculum of 2.0×10⁶ cells of an isolate of Staphylococcus aureus in the presence of a swatch (0.25 cm²) of the fabric from a BAND-AID™ adhesive bandage (Johnson & Johnson). Swatch (BAND-AID™) modified by contacting with a dispersion in water of the lipidated polyanionic molecule designated FSL-biotin, crystal violet and silver nitrate (FSL-BCA). Untreated swatch (BAND-AID™) with inoculum (positive growth control) (PC). A volume of 0 μL, 5 μL (S 5), 20 μL (S 20) and 50 μL (S 50) of serum included.

FIG. 27. Growth (log cfu) in Mueller Hinton Broth of an inoculum of 2.0×10⁶ cells of an isolate of Staphylococcus epidermis in the presence of a swatch (0.25 cm²) of the fabric from a BAND-AID™ adhesive bandage (Johnson & Johnson). Swatch (BAND-AID™) modified by contacting with a dispersion in water of the lipidated polyanionic molecule designated FSL-biotin, crystal violet and silver nitrate (FSL-BCA). Untreated swatch (BAND-AID™) with inoculum (positive growth control) (PC). A volume of 0 μL, 5 μL (S 5), 20 μL (S 20) and 50 μL (S 50) of serum included.

FIG. 28. Growth (log cfu) in Mueller Hinton Broth of an inoculum of 1.6×10⁶ cells of an isolate of Pseudomonas aeruginosa in the presence of a swatch (0.25 cm²) of the fabric from a BAND-AID™ adhesive bandage (Johnson & Johnson). Swatch (BAND-AID™) modified by contacting with a dispersion in water of the lipidated polyanionic molecule designated FSL-biotin, crystal violet and silver nitrate (FSL-BCA). Untreated swatch (BAND-AID™) with inoculum (positive growth control) (PC). A volume of 0 μL, 5 μL (S 5), 20 μL (S 20) and 50 μL (S 50) of serum included.

FIG. 29. Growth (log cfu) in Mueller Hinton Broth of an inoculum of 4.1×10⁶ cells of an isolate of Escherichia coli in the presence of a swatch (0.25 cm²) of the fabric from a BAND-AID™ adhesive bandage (Johnson & Johnson). Swatch (BAND-AID™) modified by contacting with a dispersion in water of the lipidated polyanionic molecule designated FSL-biotin, crystal violet and silver nitrate (FSL-BCA). Untreated swatch (BAND-AID™) with inoculum (positive growth control)(PC). A volume of 0 μL, 5 μL (S 5), 20 μL (S 20) and 50 μL (S 50) of serum included.

FIG. 30. A template (A) for the wells of a 24-well plate (B) showing no growth (dark blue) in columns A and B and growth (light red) in columns C and D. Wells in columns 1 and 2 contained stamped coupons of stainless steel (SS316) that had been contacted with a dispersion in water of the lipidated polyanionic molecule designated FSL-biotin, crystal violet and silver nitrate. Wells in columns 3 and 4 contained untreated stamped coupons of stainless steel (SS316). A volume of 5 μL of serum was added to each of the wells in row 4. A volume of 20 μL of serum was added to each of the wells in row 3. A volume of 50 μL of serum was added to each of the wells in row 2. Wells were inoculated with 2.0×10⁶ cells of an isolate of Staphylococcus aureus.

FIG. 31. A template (A) for the wells of a 24-well plate (B) showing no growth (dark blue) in columns A and B and growth (light red) in columns C and D. Wells in columns 1 and 2 contained stamped coupons of stainless steel (SS316) that had been contacted with a dispersion in water of the lipidated polyanionic molecule designated FSL-biotin, crystal violet and silver nitrate. Wells in columns 3 and 4 contained untreated stamped coupons of stainless steel (SS316). A volume of 5 μL of serum was added to each of the wells in row 4. A volume of 20 μL of serum was added to each of the wells in row 3. A volume of 50 μL of serum was added to each of the wells in row 2. Wells were inoculated with 2.0×10⁶ cells of an isolate of Staphylococcus epidermis.

FIG. 32. A template (A) for the wells of a 24-well plate (B) showing no growth (dark blue) in columns A and B and growth (light red) in columns C and D. Wells in columns 1 and 2 contained stamped coupons of stainless steel (SS316) that had been contacted with a dispersion in water of the lipidated polyanionic molecule designated FSL-biotin, crystal violet and silver nitrate. Wells in columns 3 and 4 contained untreated stamped coupons of stainless steel (SS316). A volume of 5 μL of serum was added to each of the wells in row 4. A volume of 20 μL of serum was added to each of the wells in row 3. A volume of 50 μL of serum was added to each of the wells in row 2. Wells were inoculated with 1.6×10⁶ cells of an isolate of Pseudomonas aeruginosa.

FIG. 33. A template (A) for the wells of a 24-well plate (B) showing no growth (dark blue) in columns A and B and growth (light red) in columns C and D. Wells in columns 1 and 2 contained stamped coupons of stainless steel (SS316) that had been contacted with a dispersion in water of the lipidated polyanionic molecule designated FSL-biotin, crystal violet and silver nitrate. Wells in columns 3 and 4 contained untreated stamped coupons of stainless steel (SS316). A volume of 5 μL of serum was added to each of the wells in row 4. A volume of 20 μL of serum was added to each of the wells in row 3. A volume of 50 μL of serum was added to each of the wells in row 2. Wells were inoculated with 4.1×10⁶ cells of an isolate of Escherichia coli.

### DESCRIPTION

It will be understood that augmenting the antimicrobial activity imparted to the surface of a substrate includes either or both of increasing the biocidal or biostatic activity against a species of microorganism or increasing the range of species of microorganism against which the surface treatment is active. The inventors have determined that water dispersible lipid conjugated polyanionic molecules may be used in conjunction with water-soluble antimicrobial agents to impart an antimicrobial activity to a surface. Importantly, the imparted activity is retained despite washing with water or the presence of serum.

Without wishing to be bound by theory it is believed the greater antimicrobial activity observed when a lipid conjugated polyanionic molecule is used in conjunction with one or more water-soluble antimicrobial agents may be attributed to a localised concentration effect at the treated surface. In the absence of this surface effect the components of the antimicrobial composition, e.g. silver and hexamethylpararosaniline, would be anticipated to become uniformly distributed throughout the milieu contacting the surface and thereby have reduced antimicrobial activity.

The method is demonstrated here using the compounds crystal violet and silver nitrate. In addition to its antibacterial action (Adams (1967), Docampo and Moreno (1990)), crystal violet is a dye facilitating the determination of its retention at a surface despite rinsing or washing with water. As the method illustrated in the following examples appears to be dependent on nonspecific interactions between the compound and the lipid conjugated polyanionic molecule the applicability of the method to use with a range of water-dispersible antimicrobial agents is anticipated.

Another advantage of the method is that it imparts an antimicrobial activity to a surface that is stable under conditions commonly used to sterilize adhesive bandages or medical implants. Sterilisation by autoclaving will obviously be subject to the compatibility of the untreated surface with these conditions. For example, certain polymers may not tolerate temperatures of 120° C., and certain metals may not tolerate contact with steam. In these circumstances dry heat (80° C.) or irradiation may be more appropriate methods of sterilisation.

Chemistry

Acetone, benzene, chloroform, ethylacetate, methanol, toluene and o-xylene were from Chimmed (Russian Federation). Acetonitrile was from Cryochrom (Russian Federation). DMSO, DMF, CF₃COOH, Et₃N, N,N′-dicyclohexylcarbodiimide and N-hydroxysuccinimide were from Merck (Germany). Iminodiacetic acid dimethyl ester hydrochloride was from Reakhim (Russian Federation). Dowex 50X4-400 and Sephadex LH-20 were from Amersham Biosciences AB (Sweden). Silica gel 60 was from Merck (Germany). Tetraamine (H₂N—CH₂)₄C×2H₂SO₄ was synthesized as described by Litherland et al. (1938). Thin-layer chromatography was performed using silica gel 60 F254 aluminium sheets (Merck, 1.05554) with detection by charring after 7% H₃PO₄ soaking.

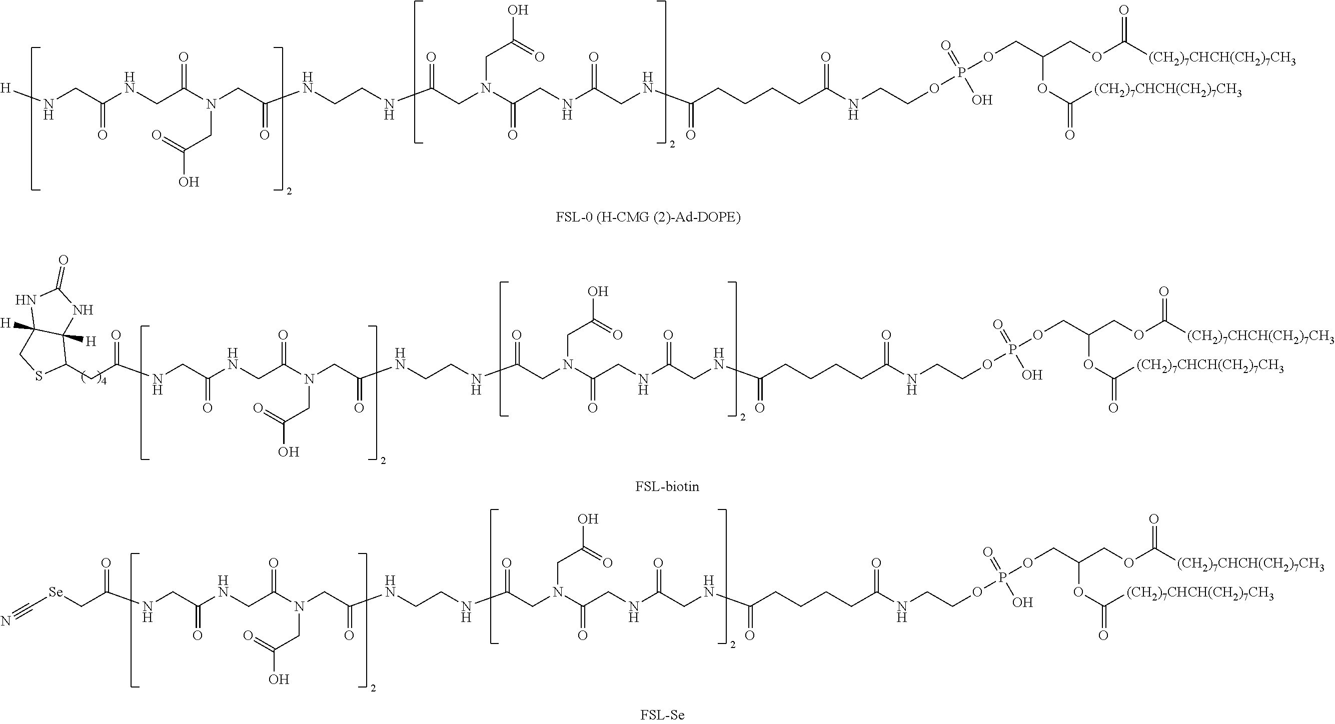

Preparation of the Constructs Designated FSL-0, FSL-Biotin and FSL-Se

To a stirred solution of (methoxycarbonylmethyl-amino)-acetic acid methyl ester hydrochloride (988 mg, 5 mmol) in DMF (15 ml) were added Boc-GlyGlyNos (3293 mg, 10 mmol) and (CH₃CH₂)₃N (3475 μL, 25 mmol) were added. The mixture was stirred overnight at room temperature and then diluted with o-xylene (70 ml) and evaporated. Flash column chromatography on silica gel (packed in toluene and eluted with ethyl acetate) resulted in a crude product. The crude product was dissolved in chloroform and washed sequentially with water, 0.5 M NaHCO₃ and saturated KCl. The chloroform extract was evaporated and the product purified on a silica gel column (packed in chloroform and eluted with 15:1 (v/v) chloroform/methanol). Evaporation of the fractions and drying under vacuum of the residue provided a colourless thick syrup. Yield 1785 mg, (95%). TLC: R_{f}=0.49 (7:1 (v/v) chloroform/methanol).

¹H NMR (500 MHz, [D₆]DMSO, 30° C.) δ, ppm: 7.826 (t, J=5.1 Hz, 1H; NHCO), 6.979 (t, J=5.9 Hz, 1H; NHCOO), 4.348 and 4.095 (s, 2H; NCH₂COO), 3.969 (d, J=5.1 Hz, 2H; COCH₂NH), 3.689 and 3.621 (s, 3H; OCH₃), 3.559 (d, J=5.9 Hz, 2H; COCH₂NHCOO), 1.380 (s, 9H; C(CH₃)₃).

To a stirred solution of {[2-(2-tert-butoxycarbonylamino-acetylamino)-acetyl]-methoxycarbonylmethyl-amino}-acetic acid methyl ester (1760 mg, 4.69 mmol) in methanol (25 ml) 0.2 M aqueous NaOH (23.5 ml) was added and the solution kept for 5 min at room temperature. The solution was then acidified with acetic acid (0.6 ml) and evaporated to dryness. Column chromatography of the residue on silica gel (packed in ethyl acetate and eluted with 2:3:1 (v/v/v) i-PrOH/ethyl acetate/water) resulted in a recovered {[2-(2-tert-butoxycarbonylamino-acetylamino)-acetyl]-methoxycarbonylmethyl-amino}-acetic acid methyl ester (63 mg, 3.4%) and target compound (1320 mg). The intermediate product was then dissolved in methanol/water/pyridine mixture (20:10:1, 30 ml) and passed through an ion exchange column (Dowex 50X4-400, pyridine form, 5 ml) to remove residual sodium cations. The column was then washed with the same solvent mixture, the eluant evaporated, the residue dissolved in chloroform/benzene mixture (1:1, 50 ml) and then evaporated and dried under vacuum. Yield of 10 was 1250 mg (74%), white solid. TLC: R_{f}=0.47 (4:3:1 (v/v/v) i-PrOH/ethyl acetate/water).

¹H NMR (500 MHz, [D₆]DMSO, 30° C.), mixture of cis- and trans-conformers of N-carboxymethylglycine unit c.3:1. Major conformer; δ, ppm: 7.717 (t, J=5 Hz, 1H; NHCO), 7.024 (t, J=5.9 Hz, 1H; NHCOO), 4.051 (s, 2H; NCH₂COOCH₃), 3.928 (d, J=5 Hz, 2H; COCH₂NH), 3.786 (s, 2H; NCH₂COOH), 3.616 (s, 3H; OCH₃), 3.563 (d, J=5.9 Hz, 2H; COCH₂NHCOO), 1.381 (s, 9H; C(CH₃)₃) ppm; minor conformer, δ=7.766 (t, J=5 Hz, 1H; NHCO), 7.015 (t, J=5.9 Hz, 1H; NHCOO), 4.288 (s, 2H; NCH₂COOCH₃), 3.928 (d, J=5 Hz, 2H; COCH₂NH), 3.858 (s, 2H; NCH₂COOH), 3.676 (s, 3H; OCH₃), 3.563 (d, J=5.9 Hz, 2H; COCH₂NHCOO), 1.381 (s, 9H; C(CH₃)₃).

To an ice-cooled stirred solution of {[2-(2-tert-butoxycarbonylamino-acetylamino)-acetyl]-methoxycarbonylmethyl-amino}-acetic acid (1200 mg, 3.32 mmol) and N-hydroxysuccinimide (420 mg, 3.65 mmol) in DMF (10 ml) was added N,N′-dicyclohexylcarbodiimide (754 mg, 3.65 mmol). The mixture was stirred at 0° C. for 30 min, then for 2 hours at room temperature. The precipitate of N,N′-dicyclohexylurea was filtered off, washed with DMF (5 ml), and filtrates evaporated to a minimal volume. The residue was then agitated with (CH₃CH₂)₂O (50 ml) for 1 hour and an ether extract removed by decantation. The residue was dried under vacuum providing the active ester (1400 mg, 92%) as a white foam. TLC: R_{f}=0.71 (40:1 (v/v) acetone/acetic acid).

¹H NMR (500 MHz, [D₆]DMSO, 30° C.), mixture of cis- and trans-conformers of N-carboxymethylglycine unit c. 3:2.

Major conformer; δ, ppm: 7.896 (t, J=5.1 Hz, 1H; NHCO), 6.972 (t, J=5.9 Hz, 1H; NHCOO), 4.533 (s, 2H; NCH₂COON), 4.399 (s, 2H; NCH₂COOCH₃), 3.997 (d, J=5.1 Hz, 2H; COCH₂NH), 3.695 (s, 3H; OCH₃), 3.566 (d, J=5.9 Hz, 2H; COCH₂NHCOO), 1.380 (s, 9H; C(CH₃)₃).

Minor conformer; δ, ppm: 7.882 (t, J=5.1 Hz, 1H; NHCO), 6.963 (t, J=5.9 Hz, 1H; NHCOO), 4.924 (s, 2H; NCH₂COON), 4.133 (s, 2H; NCH₂COOCH₃), 4.034 (d, J=5.1 Hz, 2H; COCH₂NH), 3.632 (s, 3H; OCH₃), 3.572 (d, J=5.9 Hz, 2H; COCH₂NHCOO), 1.380 (s, 9H; C(CH₃)₃).

The active ester (1380 mg) was dissolved in DMSO to provide a volume of 6 ml and used as a 0.5 M solution (stored at −18° C.).

To the stirred solution of (methoxycarbonylmethyl-amino)-acetic acid methyl ester hydrochloride (988 mg, 5 mmol) in DMF (15 ml) Boc-GlyGlyNos (3293 mg, 10 mmol) and Et₃N (3475 μl, 25 mmol) were added. The mixture was stirred overnight at room temperature (r.t.), then diluted with o-xylene (70 ml) and evaporated. Flash column chromatography on silica gel (packed in toluene and eluted with ethyl acetate) resulted in crude product. The crude product was dissolved in chloroform and washed sequentially with water, 0.5 M NaHCO₃ and saturated KCl. The chloroform extract was evaporated, and the product was purified on a silica gel column (packed in chloroform and eluted with chloroform/methanol 15:1). Evaporation of fractions and vacuum drying of residue resulted in a product containing colorless thick syrup (1785 mg, 95%). TLC: R_{f}=0.49 (chloroform/methanol 7:1).

¹H NMR (500 MHz, [D₆]DMSO, 30° C.) 5=7.826 (t, J=5.1 Hz, 1H; NHCO), 6.979 (t, J=5.9 Hz, 1H; NHCOO), 4.348 and 4.095 (s, 2H; NCH₂COO), 3.969 (d, J=5.1 Hz, 2H; COCH₂NH), 3.689 and 3.621 (s, 3H; OCH₃), 3.559 (d, J=5.9 Hz, 2H; COCH₂NHCOO), 1.380 (s, 9H; CMe₃) ppm.

To the stirred solution of {[2-(2-tert-butoxycarbonylamino-acetylamino)-acetyl]-methoxycarbonylmethyl-amino}-acetic acid methyl ester (1760 mg, 4.69 mmol) in methanol (25 ml) 0.2 M aqueous NaOH (23.5 ml) was added. The solution was kept for 5 min at r.t., then acidified with acetic acid (0.6 ml) and evaporated to dryness. Column chromatography of the residue on silica gel (packed in ethyl acetate and eluted with iPrOH/ethyl acetate/water (2:3:1)) resulted in recovered product (63 mg, 3.4%) and crude target compound (1320 mg). The crude target compound was dissolved in methanol/water/pyridine mixture (20:10:1, 30 ml) and passed through an ion-exchange column (Dowex 50X4-400, pyridine form, 5 ml) to remove residual Na cations. The column was washed with the same mixture, eluant evaporated, dissolved in chloroform/benzene mixture (1:1, 50 ml) then evaporated and dried in vacuum to provide a yield of pure (10) was 1250 mg (74%), white solid. TLC: R_{f}=0.47 (iPrOH/ethyl acetate/water (4:3:1)).

¹H NMR (500 MHz, [D₆]DMSO, 30° C.) of mixture of cis- and trans-conformers of N-carboxymethyl-glycine unit c.3:1.

Major conformer: δ=7.717 (t, J=5 Hz, 1H; NHCO), 7.024 (t, J=5.9 Hz, 1H; NHCOO), 4.051 (s, 2H; NCH₂COOMe), 3.928 (d, J=5 Hz, 2H; COCH₂NH), 3.786 (s, 2H; NCH₂COOH), 3.616 (s, 3H; OCH₃), 3.563 (d, J=5.9 Hz, 2H; COCH₂NHCOO), 1.381 (s, 9H; CMe₃) ppm.

Minor conformer: δ=7.766 (t, J=5 Hz, 1H; NHCO), 7.015 (t, J=5.9 Hz, 1H; NHCOO), 4.288 (s, 2H; NCH₂COOMe), 3.928 (d, J=5 Hz, 2H; COCH₂NH), 3.858 (s, 2H; NCH₂COOH), 3.676 (s, 3H; OCH₃), 3.563 (d, J=5.9 Hz, 2H; COCH₂NHCOO), 1.381 (s, 9H; CMe₃) ppm.

To an ice-cooled stirred solution of {[2-(2-tert-butoxycarbonylamino-acetylamino)-acetyl]-methoxycarbonylmethyl-amino}-acetic acid (1200 mg, 3.32 mmol) and N-hydroxysuccinimide (420 mg, 3.65 mmol) in DMF (10 ml) N,N′-dicyclohexylcarbodiimide (754 mg, 3.65 mmol) was added. The mixture was stirred at 0° C. for 30 min, then for 2 h at r.t. The precipitate of N,N′-dicyclohexylurea was filtered off, washed with DMF (5 ml) and the filtrates evaporated to a minimal volume. The residue was agitated with Et₂O (50 ml) for 1 h. An ether extract was removed by decantation, and the residue dried in polyanionic maleimide-lipid construct designated Mal-(CH₂)₂CO-CMG(2)-Ad-DOPE.

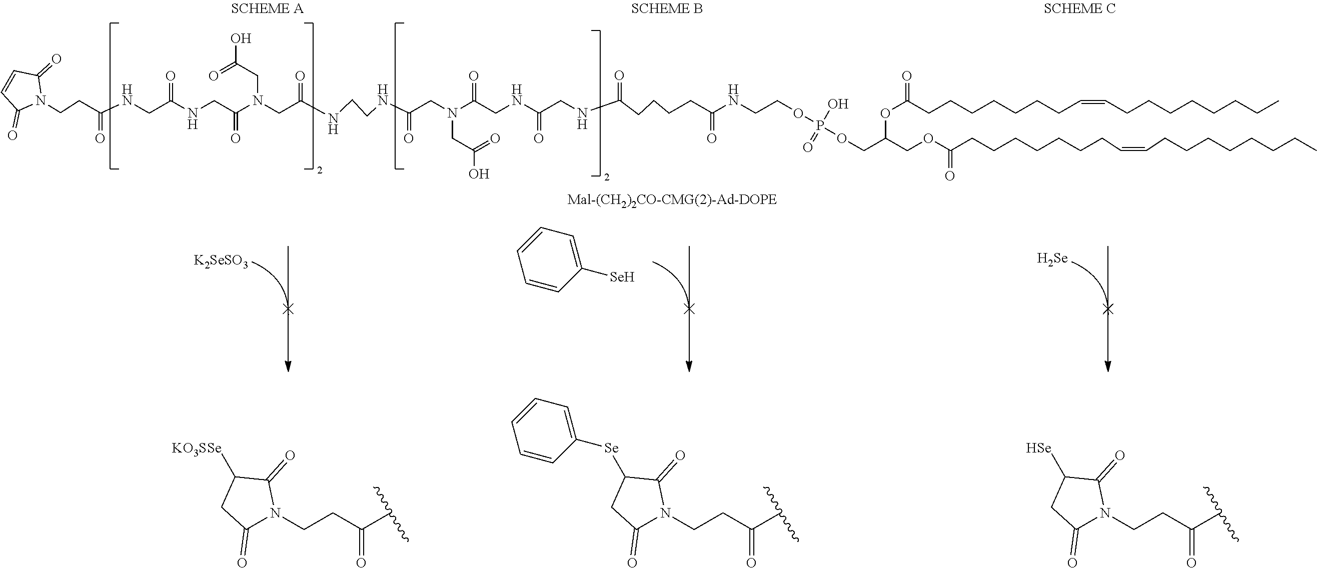

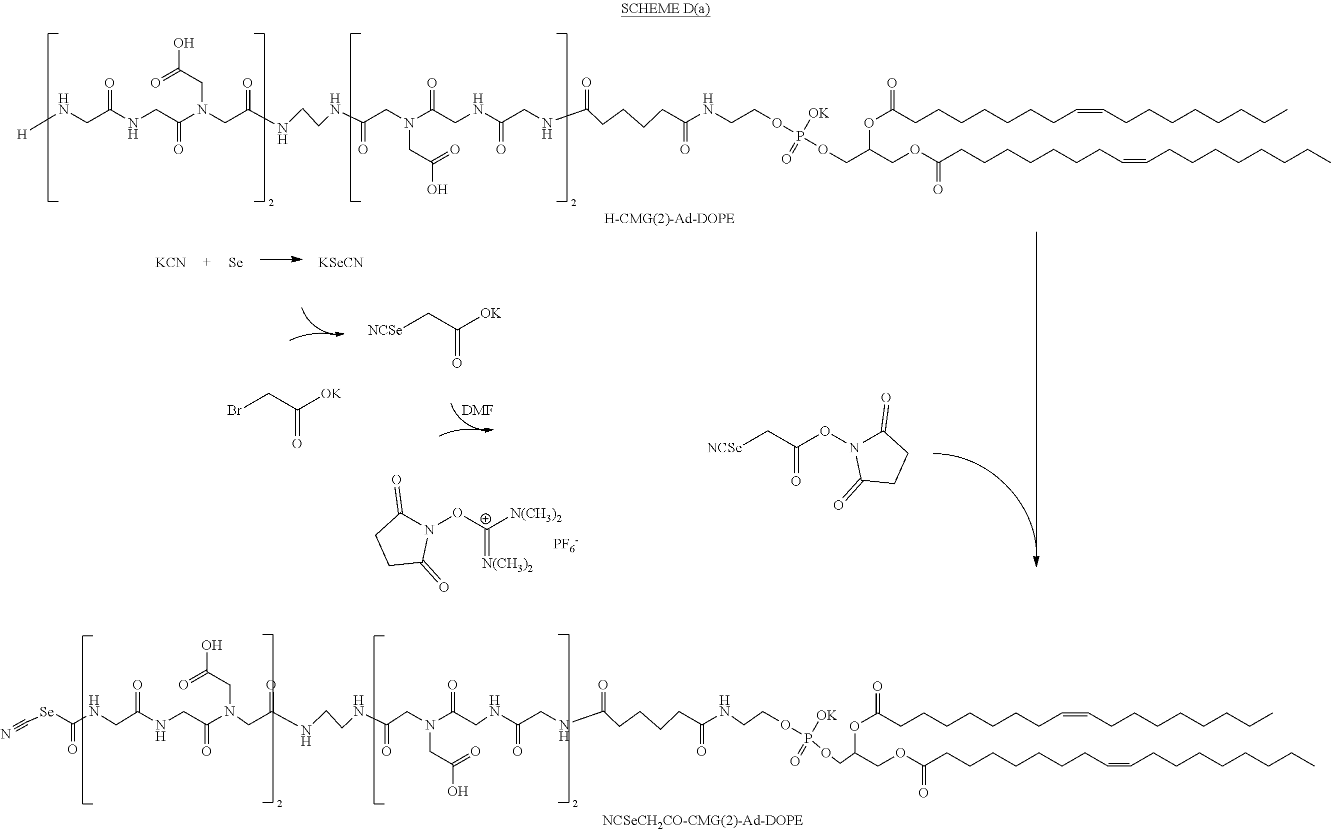

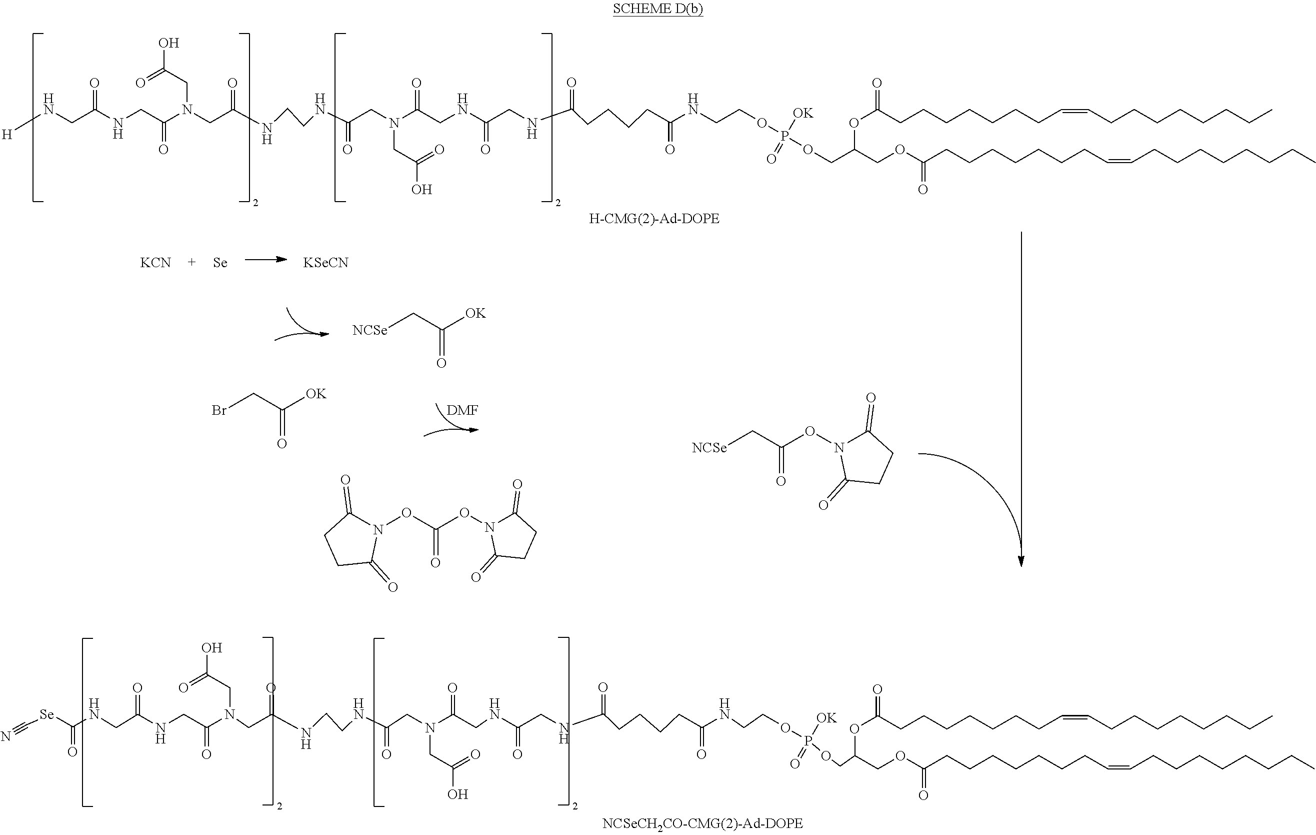

vacuum to yield the target compound (1400 mg, 92%) as a white foam. TLC: R_{f}=0.71 (acetone/acetic acid 40:1).

¹H NMR (500 MHz, [D₆]DMSO, 30° C.), mixture of cis- and trans-conformers of N-carboxymethyl-glycine unit c. 3:2.

Major conformer: δ=7.896 (t, J=5.1 Hz, 1H; NHCO), 6.972 (t, J=5.9 Hz, 1H; NHCOO), 4.533 (s, 2H; NCH₂COON), 4.399 (s, 2H; NCH₂COOMe), 3.997 (d, J=5.1 Hz, 2H; COCH₂NH), 3.695 (s, 3H; OCH₃), 3.566 (d, J=5.9 Hz, 2H; COCH₂NHCOO), 1.380 (s, 9H; CMe₃) ppm.

Minor conformer: δ=7.882 (t, J=5.1 Hz, 1H; NHCO), 6.963 (t, J=5.9 Hz, 1H; NHCOO), 4.924 (s, 2H; NCH₂COON), 4.133 (s, 2H; NCH₂COOMe), 4.034 (d, J=5.1 Hz, 2H; COCH₂NH), 3.632 (s, 3H; OCH₃), 3.572 (d, J=5.9 Hz, 2H; COCH₂NHCOO), 1.380 (s, 9H; CMe₃) ppm.

Attempts to prepare a cyanoselenide-lipid construct via an addition reaction between the maleimide-lipid construct designated Mal-(CH₂)₂CO-CMG(2)-Ad-DOPE and potassium selenosulfite (K₂SeSo₃) [SCHEME A], selenophenol (PhSeH) [SCHEME B] and hydrogen selenide (H₂Se) [SCHEME C] were unsuccessful. With hindsight the failure to obtain a stable seleno-Bunte salt according to SCHEME A is at least in part predictable from the disclosure of the chemical behaviour of their sulfur analogues in the publication of Distler (1967). Both the attempted Michael additions of phenylselenide and hydrogen selenide in protic media according to SCHEME B and SCHEME C, respectively, yielded a product with a reduced maleimide double bond, as opposed to the desired selenylsuccinimides. Formation of selenylsuccinimides in quantitative yield has been disclosed in the publication of Numeo et al (1981). However, the disclosed use of anhydrous ether is incompatible with the use of the

It was subsequently discovered that the cyanoselenide-lipid construct designated NCSeCH₂CO-CMG(2)-Ad-DOPE could be successfully prepared via an activated 2-selenocyanatoacetic acid (NC—Se—CH₂COOH). The activated NC—Se—CH₂COOH was reacted with the lipid construct H-CMG(2)-Ad-DOPE according to SCHEME D(a) or SCHEME D(b). The prepared construct was stored in the dark under an inert atmosphere. Potassium selenocyanate was selected as the reagent of choice as it could readily be activated as an N-hydroxysuccinimide (NHS) ester according to SCHEME D(a) or (b) or mixed anhydride according to SCHEME D(c). Potassium selenocyanoacetate (NCSeCH₂COOK) was synthesized from freshly prepared solutions of potassium selenocyanate (KSeCN) and potassium bromoacetate (BrCH₂COOK) according to the procedures disclosed in the publication of Klauss (1970). The synthesized NCSeCH₂COOK was stored in a vacuum desiccator over potassium hydroxide (KOH) pellets in the dark prior to activation. For activation the potassium selenocyanoacetate (156 mg, 0.77 mmol) was added in one portion to a solution of N,N,N′,N′-tetramethyl-O—(N-succinimidyl)uraniumhexafluorophosphate (HSTU) (IRIS, Germany) (212 mg, 0.59 mmol) in 1 mL DMF while a gentle flow of dry argon via a PTFE capillary was bubbling through. The slurry thus obtained was stirred in this way for 30 minutes during which the initial solid changed to a more dense crystalline precipitate (KPF₆). The reaction mixture was sonicated for 1 to 2 minutes and combined with the construct designated H-CMG(2)-Ad-DOPE (110 mg, 0.06 mmol) dissolved in 1 mL of 20% IPA followed by 100 μL 1N KHCO₃. A sticky solid (presumably NCSeCH₂COOSu) that precipitated immediately, was dissolved by dropwise addition of 30% IPA (circa 1.6 mL) with sonication and the reaction mixture was magnetically stirred for 3 hours at room temperature keeping pH in the range 8.0 to 8.5 (TLC control: Solvents were evaporated in vacuum and dry residue was triturated with 3 mL of acetonitrile with sonication until fine slurry formed and then transferred into Eppendorf tubes (2×2.2 mL), centrifuged and the solids washed 4 times consecutively with neat IPA and MeCN (2 mL of each, brief sonication followed by centrifugation). The wet solids were dissolved in 3.5 mL of 30% IPA-water and lyophilized to constant weight. 111 mg (92%) of the cyanoselenide-lipid construct designated NCSeCH₂CO-CMG(2)-Ad-DOPE were obtained as a reddish amorphous powder. R_{f}˜0.5, CHCl₃/methanol/water 2:6:1 (v/v); TLC aluminium sheets Silica gel 60 F254 (Merck 1.05554). It is noted that mass spectroscopy did not appear suitable for the characterization of this construct. Only peaks of Se-free fragments could be detected.

The lipidated polyanionic molecule designated FSL-biotin is prepared by biotinylation of FSL-0. The preparation of other lipidated polycarboxylic molecules is described in the specification accompanying U.S. patent application Ser. No. 15/350,792 [publ. no US 2017/0218027 A1] the disclosures of which are hereby incorporated by reference.

Biology

Monitoring Growth of Bacteria and Formation of Biofilms

Swatches (1 cm diameter circular disks) were cut from the fabric portion of commercially available adhesive bandages (BAND-AID™ Quilt-Aid Technology, Johnson & Johnson). Individual swatches were each dipped into a 0.13 mM, 0.25 mM or 1 mM dispersion in deionized (DI) water of the construct designated FSL-Se, or water alone (control). The dipped swatches were then air dried at 80° C. Four replicates of each treatment, including a control, were prepared and two swatches from each group of replicates individually washed by allowing deionized water to flow over the surface for thirty seconds.

Individual unwashed and washed swatches from each treatment group were then placed at the base of separate wells in a 24-well cell culture plate.

Frozen stock cultures of Staphylococcus aureus were streaked on Columbia sheep blood agar plates (Fort Richard Laboratories) and the plates incubated at 37° C. until separate colonies developed. Two to three colonies were transferred to a volume of 10 mL of 21 g/L Muller-Hinton (MH) broth, vortexed, and the turbidity measured at 600 nm. The MH broth was diluted with a further volume so that the turbidity of the suspension provided an optical density of 0.08. A 1000-fold dilution of this suspension was then used as an inoculum.

A volume of 10 μL of the inoculum corresponding to approximately 460 colony forming units (cfu) was transferred to the surface of each of the swatches (excluding the controls) and incubated at room temperature for one hour. A volume of 60 μL of MH broth was then dispensed into each well and the plate incubated at room temperature for a further two hours before adding a volume of 1 mL consisting of 100 μL of a 0.02% (w/v) solution in deionized water of resazurin sodium salt (Sigma-Aldrich) and 900 μL of MH broth. The colour change (if any) observed in each well was observed after incubation of the plate for 24 hours at 37° C. (Elshikh et al (2016), Mann and Markham (1998), Montoro et al (2005), Pettit et al (2005) and Sarker et al (2007)).

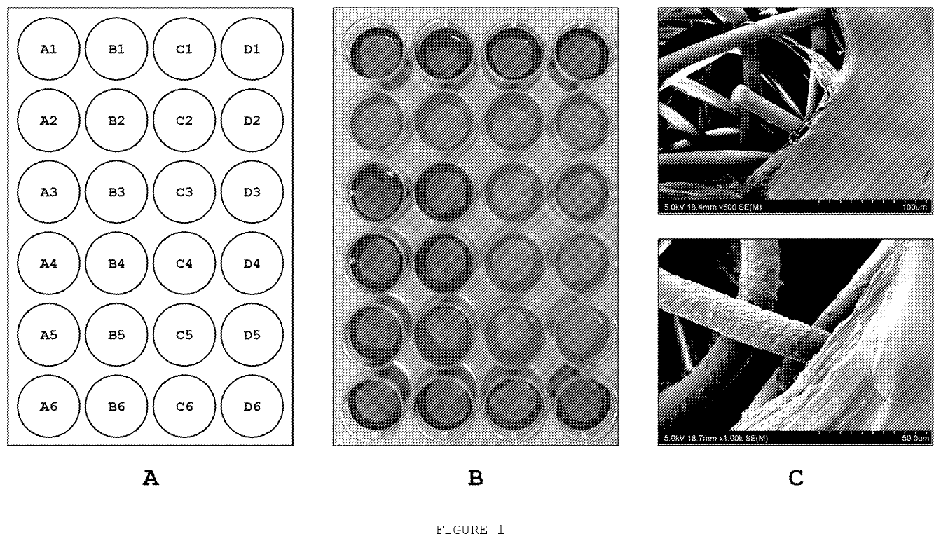

Incubated swatches were recovered from each well, washed by dipping sterile deionised water and then incubated for 2 hours in a solution of 2.5% (w/v) glutaraldehyde. The incubated swatches were once more washed by dipping in sterile deionised water, dried, sputter coated with platinum for 60 seconds and observed under an electron microscope at 5 kV. The observations are presented in FIG. 1.

Retention of Crystal Violet by Treated Substrates

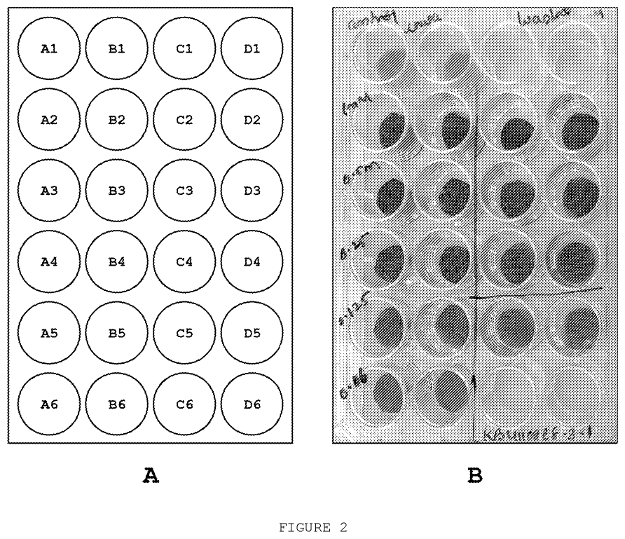

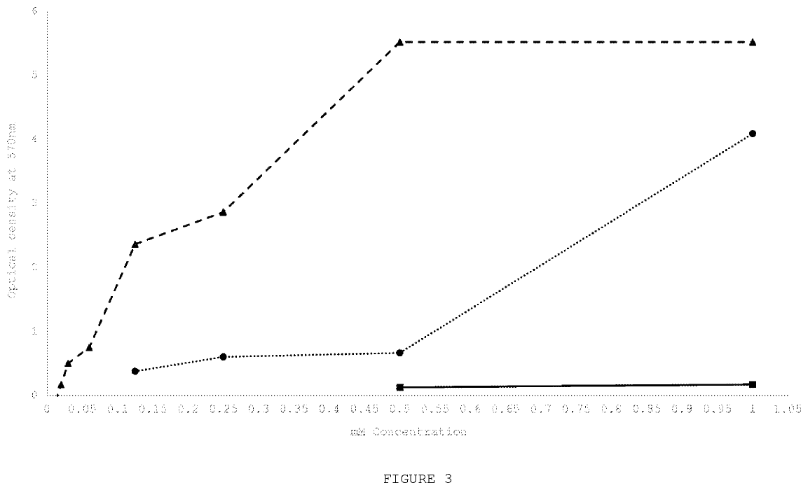

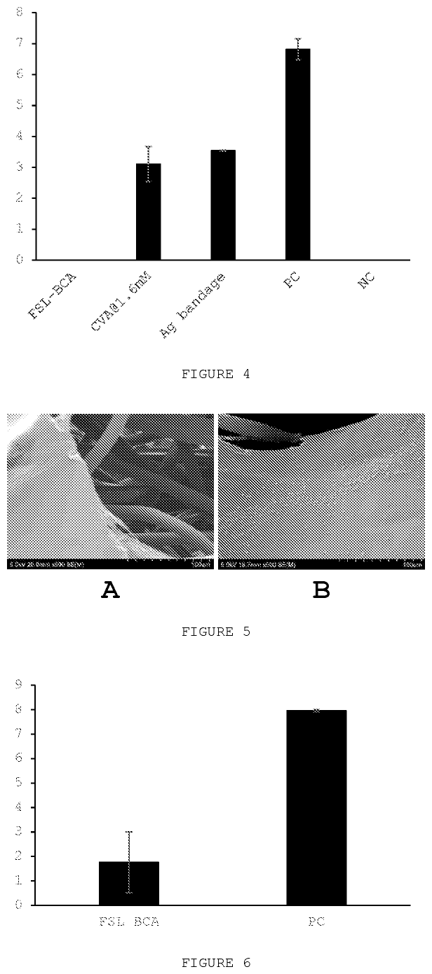

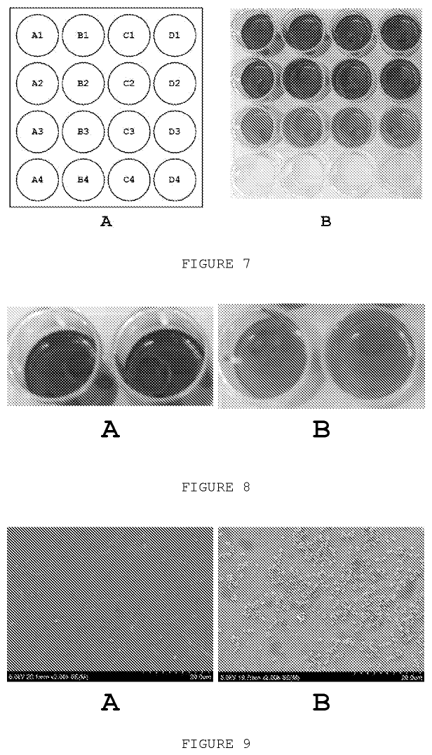

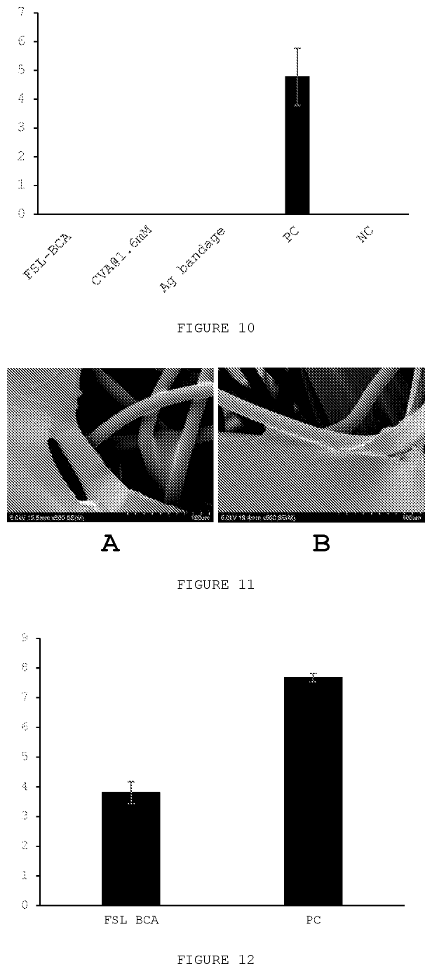

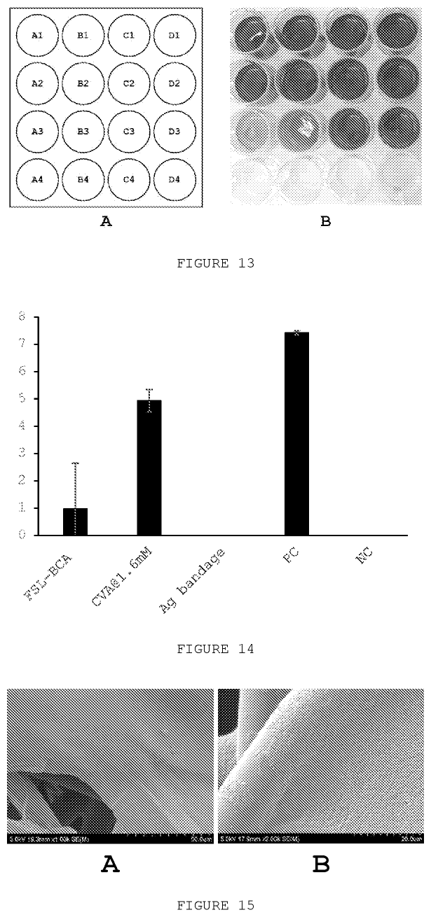

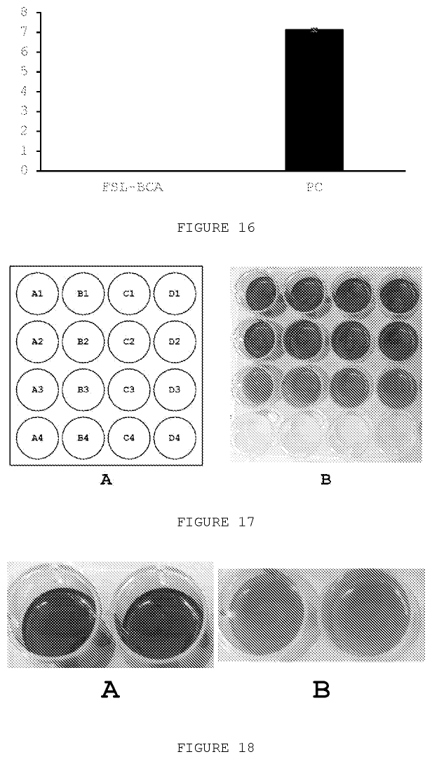

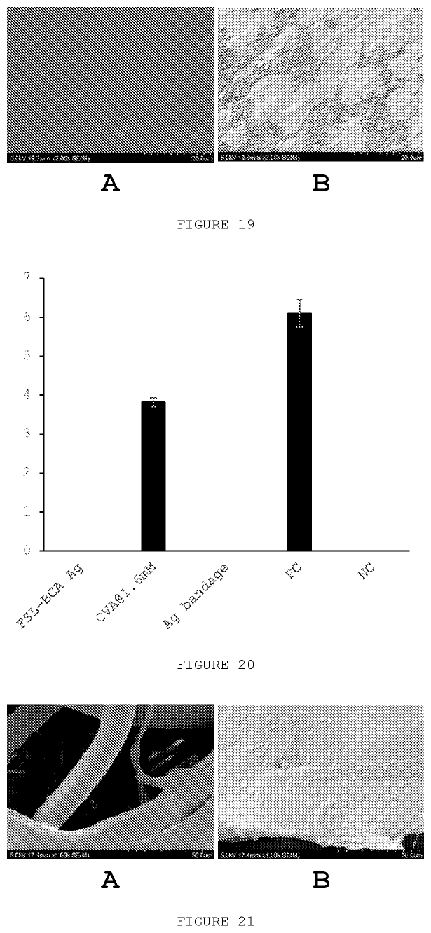

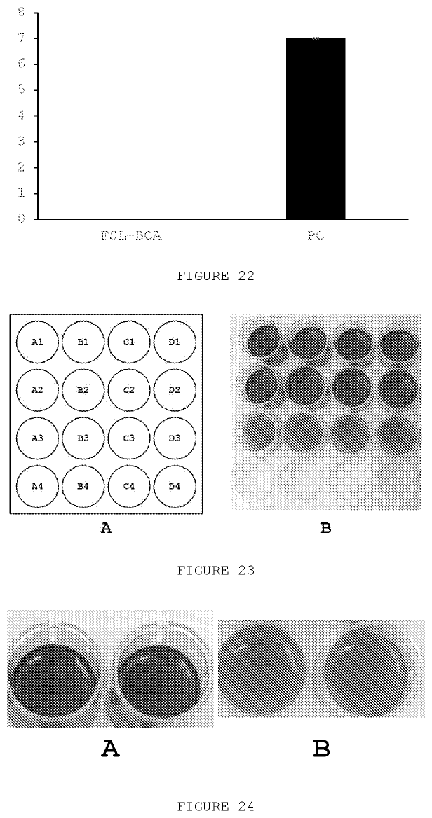

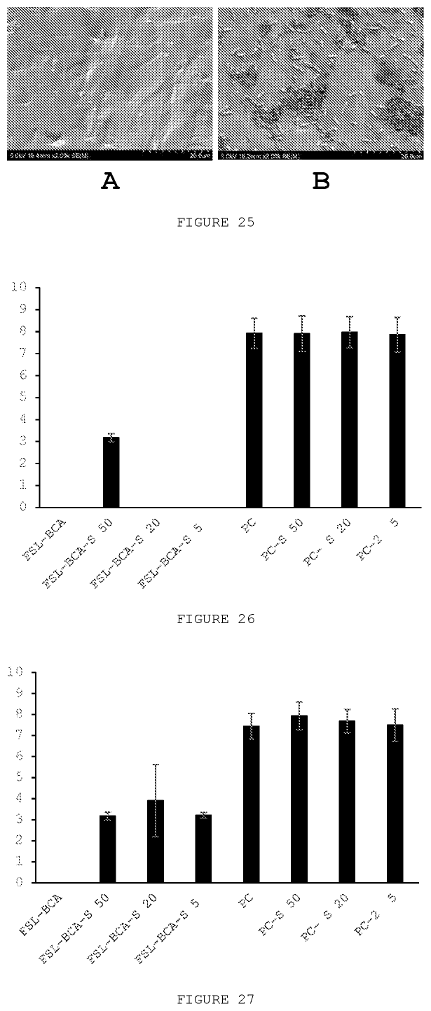

Swatches (1 cm diameter circular disks) were cut from the fabric portion of commercially available adhesive bandages (BAND-AID™ Quilt-Aid Technology, Johnson & Johnson). A volume of 50 μL of a dispersion in deionized water of a construct (FSL-Se, FSL-biotin or FSL-0) or water alone (control) was dispensed onto the surface of individual swatches. The swatches were air dried at 80° C. before a volume of 100 μL of a 0.04% (w/v) solution of crystal violet in deionized water was dispensed onto the surface of each treated and untreated (control) swatch, incubated undisturbed at room temperature for 10 minutes, before rinsing with deionized water to remove the unretained crystal violet. Observations of the swatches following rinsing with water are presented in FIG. 2. The retained crystal violet was recovered by washing each swatch with a volume of 200 μL of 95% (v/v) ethanol and the absorbance of 570 nm of a volume of 100 μL of the wash measured. The measured absorbances are recorded in FIG. 3.

Augmented Antibacterial Surface Treatment

Swatches (1 cm diameter circular disks) were cut from the fabric portion of commercially available adhesive bandages (BAND-AID™ Quilt-Aid Technology, Johnson & Johnson). Volumes of 50 μL were dispended onto the surface of the swatches and the swatches allowed to air dry undisturbed at 80° C. The volumes consisted of dispersions or solutions in deionized water of 0.04% (w/v) crystal violet, 0.25 mM or 0.13 mM of the construct designated FSL-Se or the construct designated FSL-0, or water alone. A further volume of 100 μL of 0.04% (w/v) crystal violet in deionized water was dispensed onto the surface of the dried treated swatches and incubated undisturbed at room temperature for 10 minutes before rinsing as before with sterile deionized water.

Frozen stock cultures of bacterial isolates (Staphylococcus aureus, Staphylococcus epidermis, Escherichia coli or Pseudomonas aeruginosa) were streaked on Columbia sheep blood agar plates (Fort Richard Laboratories) and the plates incubated at 37° C. for 24 hours when separate colonies had developed. Two to three colonies were then transferred to a volume of 10 mL of 21 g/L Muller-Hinton (MH) broth, vortexed and the turbidity measured at 600 nm. The MH broth was diluted with a further volume so that the turbidity provided an optical density of 0.20 for the suspensions of Staphylococcus aureus and Staphylococcus epidermis and 0.08 for the suspensions of Escherichia coli and Pseudomonas aeruginosa. The diluted suspensions (around 10⁸ cfu/mL) were serially diluted.

TABLE 1
Growth of an isolate of Staphylococcus aureus on treated and
untreated substrate as monitored by the colour change from dark blue
(no growth) to light red (growth) via purple (reduced growth) of
resazurin containing MH broth. Substrate was treated with crystal
violet alone or in combination with a construct (FSL-Se or FSL-0)
at the concentrations (mM) indicated. Substrates
were challenged with an inoculum of the bacterium at
the concentration (cfu) indicated. Hyphens indicate not tested.

| Challenge | Crystal | 0.25 mM | 0.13 mM | 0.25 mM | 0.13 mM |
|---|---|---|---|---|---|
| (cfu) | violet | FSL-0 | FSL-0 | FSL-Se | FSL-Se |
| >10⁵ | Reduced | No | No growth | No growth | No |
| | growth | growth | | | growth |
| 1.5 × 10⁴ to 10⁵ | Reduced | No | — | No growth | No |
| | growth | growth | | | growth |
| 10⁴ to 1.5 × 10⁴ | — | — | No growth | — | — |
| 5 × 10³ to 10⁴ | — | — | — | — | — |
| 10³ to 5 × 10³ | Reduced | No | No growth | No growth | No |
| | growth | growth | | | growth |
| 500 to 1,000 | No growth | — | No growth | — | — |

TABLE 2
Growth of an isolate of Staphylococcus epidermis on treated and
untreated substrate as monitored by the colour change from dark blue
(no growth) to light red (growth) via purple (reduced growth) of
resazurin containing MH broth. Substrate was treated with crystal
violet alone or in combination with a construct (FSL-Se or FSL-0)
at the concentrations (mM) indicated. Substrates were
challenged with an inoculum of the bacterium at
the concentration (cfu) indicated. Hyphens indicate not tested.

| Challenge | Crystal | 0.25 mM | 0.13 mM | 0.25 mM | 0.13 mM |
|---|---|---|---|---|---|
| (cfu) | violet | FSL-0 | FSL-0 | FSL-Se | FSL-Se |
| >10⁵ | Reduced | No | Growth | No growth | No |
| | growth | growth | | | growth |
| 1.5 × 10⁴ to 10⁵ | — | — | No growth | — | — |
| 10⁴ to 1.5 × 10⁴ | Reduced | No | — | No growth | No |
| | growth | growth | | | growth |
| 5 × 10³ to 10⁴ | — | — | — | — | — |
| 10³ to 5 × 10³ | Reduced | No | — | No growth | No |
| | growth | growth | | | growth |
| 500 to 1,000 | — | — | — | — | — |

TABLE 3
Growth of an isolate of Staphylococcus pseudomonas aeruginosa on
treated and untreated substrate as monitored by the colour change
from dark blue (no growth) to light red (growth) via purple (reduced
growth) of resazurin containing MH broth. Substrate was treated
with crystal violet alone or in combination with a construct
(FSL-Se or FSL-0) at the concentrations (mM) indicated.
Substrates were challenged with an inoculum of
the bacterium at the concentration (cfu) indicated. Hyphens
indicate not tested.

| Challenge | Crystal | 0.25 mM | 0.13 mM | 0.25 mM | 0.13 mM |
|---|---|---|---|---|---|
| (cfu) | violet | FSL-0 | FSL-0 | FSL-Se | FSL-Se |
| >10⁵ | Growth | Reduced | Reduced | Reduced | Reduced |
| | | growth | growth | growth | growth |
| 1.5 × 10⁴ to 10⁵ | Growth | — | Reduced | — | — |
| | | | growth | | |
| 10⁴ to 1.5 × 10⁴ | — | — | — | — | — |
| 5 × 10³ to 10⁴ | Growth | — | Reduced | — | — |
| | | | growth | | |
| 10³ to 5 × 10³ | Growth | No | Reduced | Reduced | Reduced |
| | | growth | growth | growth | growth |
| 500 to 1,000 | — | No | — | Reduced | Reduced |
| | | growth | | growth | growth |
| <500 | Growth | — | No growth | — | — |

Individual treated swatches from each treatment group were placed at the base of separate wells in a 96-well cell culture plate. Volumes of 10 μL of serial dilutions of the suspensions of bacteria were dispensed onto the surface of the swatches (excluding the negative control). The inoculated swatches were then incubated undisturbed at room temperature for 10 minutes before dispensing into each well a volume of 50 μL of sterile deionized water and 150 μL of MH broth containing 0.02% resazurin sodium salt (Sigma-Aldrich).

TABLE 4
Growth of an isolate of Escherichia coli on treated and untreated
substrate as monitored by the colour change from dark
blue (no growth) to light red (growth) via purple (reduced growth)
of resazurin containing MH broth. Substrate was treated
with crystal violet alone or in combination with a construct
(FSL-Se or FSL-0) at the concentrations (mM) indicated.
Substrates were challenged with an inoculum of the bacterium
at the concentration (cfu) indicated. Hyphens indicate not tested.

| Challenge | Crystal | 0.25 mM | 0.13 mM | 0.25 mM | 0.13 mM |
|---|---|---|---|---|---|
| (cfu) | violet | FSL-0 | FSL-0 | FSL-Se | FSL-Se |
| >10⁵ | Growth | Reduced | Growth | No | Reduced |
| | | growth | | growth | growth |
| 1.5 × 10⁴ to 10⁵ | Growth | — | No | — | — |
| | | | growth | | |
| 10⁴ to 1.5 × 10⁴ | — | No | — | No | No |
| | | growth | | growth | growth |
| 5 × 10³ to 10⁴ | Growth | — | No | — | — |
| | | | growth | | |
| 10³ to 5 × 10³ | — | No | — | No | No |
| | | growth | | growth | growth |
| 500 to 1,000 | Growth | — | No | — | — |
| | | | growth | | |
| <500 | — | — | — | — | — |

The colour change (if any) observed in each well was observed after incubation of the plate for 24 hours at 37° C. Observations are recorded in Tables 1, 2, 3 and 4.

Preparation of negatively charged silver nanoparticles Negatively charged nanoparticles of silver (AgNPs) were prepared according to the following method. Quantities of 2 g glucose and 1 g polyvinylpyrrolidone (PVP) were dissolved in water and the resultant volume heated to 90° C. A quantity of 0.5 g of silver nitrate (AgNO₃) was dissolved in a separate volume of 1 mL water and the two volumes then rapidly mixed. The mixed volume was maintained at a temperature of 90° C. for a period of time of one hour before cooling to room temperature. The AgNPs were collected by repeated (3 times) centrifugation at 14,000 rpm for a period of time of 60 minutes and resuspension in deionised filtered water (Milli-Q) to remove excess oxidation products, PVP and silver ion (Ag⁺). The washed suspension of AgNPs was stored in the dark at 4° C. The AgNPs were characterised by electron dispersive spectroscopy (EDS) and UV-Vis absorbance scanning. Zeta potential analysis confirmed the particle charge to be negative and dynamic light scattering (DLS) indicated the median particle size to be around 120 nm.

AgNP Augmented Antibacterial Surface Treatment

Swatches (5×5 mm squares) were cut from the fabric portion of commercially available adhesive bandages (BAND-AID™, Quilt-Aid Technology, Johnson & Johnson). A volume of 50 μL of a 0.13 mM dispersion of one of the following constructs was pipetted onto the surface of a swatch: FSL-biotin, FSL-0, FSL-HA_{17 kDa}, FSL-Se and FSL-spm.

The swatches were air-dried at a temperature of 80° C. prior to washing (3 times) with sterile, filtered, deionised water (Milli-Q). A volume of 100 μL of 0.04% crystal violet was pipetted onto the surface of each treated swatch and incubated at room temperature for a period of time of 10 minutes before washing (6 times) with sterile, filtered, deionised water (Milli-Q) and air-drying at a temperature of 80° C. A volume of 50 μL of the suspension of AgNPs prepared as described above was then pipetted onto the surface of each treated square and incubated at room temperature for a period of time of 10 minutes before washing (3 times) with sterile, filtered, deionised water (Milli-Q) and air-drying at 80° C. for a period of time of 10 minutes.

Controls were prepared without contacting the swatches with constructs or without washing following contacting the swatches with construct. Individual treated squares (including controls) were placed in the bottom of individual wells of a 96-well plate and inoculated with a volume of 10 μL of a serial dilution of a log phase culture in Mueller Hinton (MH) broth of one of the following bacteria:
Staphylococcus aureus
Staphylococcus epidermis
Escherichia coli
Pseudomonas aeruginosa

The inoculated swatches were then incubated for 10 minutes before dispensing into each well of the plate a volume of 50 μL of sterile, filtered, deionised water (Milli-Q) and 150 μL of MH broth containing 0.00266% (w/v) resazurin and incubating for a period of time of 24 hours at a temperature of 37° C. The observations of resulting growth (as indicated by a colour change from blue to red) from the estimated bacterial load (2.5×10⁴ or 2.5×10⁶ colony forming units (cfu)) for each treatment and species of bacterium are recorded in Tables 5 to 8.

Materials for the Following Experiments

Crystal violet (Chroma Gesellschaft™), silver nitrate (Ajax UNIVAR™), BAND-AID™ adhesive bandages (QUILT-AID™ technology, Johnson & Johnson) and stainless steel (SS316)(industrial shim 305×0.051 mm (0.002″) stamped with U shaped rod, cut out with scissors, sterilized with 70% methanol and dried at 80° C.).

Antibacterial Surface Treatments—Fabric of Adhesive Bandage

A stock culture of each clinical isolate (Staphylococcus aureus, Staphylococcus epidermidis, Pseudomonas aeruginosa and Escherichia coli) was

TABLE 5
Observations of growth (growth), inhibition of growth (inhibition) or prevention of growth (no growth) on
duplicate treated square swatches (5 × 5 mm) of adhesive bandages (BAND-AID ™, Quilt-Aid Technology,
Johnson & Johnson) following inoculation with a load of approximately 2.5 × 10⁴ or 2.5 × 10⁶ colony
forming units (cfu) of the bacterium Staphylococcus aureus and incubation at 37° C. for 24 hours.
Swatches were either washed or unwashed following application of the FSL construct.

| | unwashed | | | | washed | | | |
|---|---|---|---|---|---|---|---|---|
| Treatment | 2.53 × 10⁶ | 2.53 × 10⁶ | 2.53 × 10⁴ | 2.53 × 104⁴ | 2.53 × 10⁶ | 2.53 × 10⁶ | 2.53 × 10⁴ | 2.53 × 10⁴ |
| FSL-Se alone | Growth | Growth | Growth | Growth | Growth | Growth | Growth | Growth |
| FSL-Se with crystal | No growth | No growth | No growth | No growth | Inhibition | Inhibition | Inhibition | Inhibition |
| violet | | | | | | | | |
| FSL-Se with crystal | No growth | No growth | No growth | No growth | Inhibition | Inhibition | Inhibition | Inhibition |
| violet and AgNPs | | | | | | | | |
| FSL-spm | Growth | Growth | Growth | Growth | Growth | Growth | Growth | Growth |
| FSL-spm with crystal | Growth | Growth | Growth | Growth | Growth | Growth | Growth | Growth |
| violet | | | | | | | | |
| FSL-spm with crystal | Growth | Growth | Growth | Growth | Growth | Growth | Growth | Growth |
| violet and AgNPs | | | | | | | | |
| FSL-HA alone | Growth | Growth | Growth | Growth | Growth | Growth | Growth | Growth |
| FSL-HA with crystal | Growth | Growth | Growth | Growth | Growth | Growth | Growth | Growth |
| violet | | | | | | | | |
| FSL-HA with crystal | Growth | Growth | Growth | Growth | Growth | Growth | Growth | Growth |
| violet and AgNPs | | | | | | | | |
| FSL-0 alone | Growth | Growth | Growth | Growth | Growth | Growth | Growth | Growth |
| FSL-0 with crystal | No growth | No growth | No growth | No growth | Inhibition | Inhibition | Inhibition | Inhibition |
| violet | | | | | | | | |
| FSL-0 with crystal | No growth | No growth | No growth | No growth | Growth | Growth | No growth | No growth |
| violet and AgNPs | | | | | | | | |
| FSL-biotin alone | Growth | Growth | Growth | Growth | Growth | Growth | Growth | Growth |
| FSL-biotin with crystal | No growth | No growth | No growth | No growth | Inhibition | Inhibition | Inhibition | Inhibition |
| violet | | | | | | | | |
| FSL-biotin with crystal | No growth | No growth | No growth | No growth | Inhibition | Inhibition | Inhibition | Inhibition |
| violet and AgNPs | | | | | | | | |
| Crystal violet alone | Growth | Growth | Growth | Growth | Growth | Growth | Growth | Growth |
| Crystal violet and | Growth | Growth | Growth | Growth | Growth | Growth | Growth | Growth |
| AgNPs | | | | | | | | |
| AgNPs alone | Growth | Growth | Growth | Growth | Growth | Growth | Growth | Growth |
| Ag BAND AID | Growth | Growth | No growth | No growth | N.D. | N.D. | N.D. | N.D. |
| Positive control | Growth | Growth | Growth | Growth | Growth | Growth | Growth | Growth |
| (no treatment) | | | | | | | | |
| Negative control | No growth | No growth | No growth | No growth | No growth | No growth | No growth | No growth |
| (no treatment) | | | | | | | | |

N.D. (not determined).

TABLE 6
Observations of growth (growth), inhibition of growth (inhibition) or prevention of growth (no growth) on
duplicate treated square swatches (5 × 5 mm) of adhesive bandages (BAND-AID ™, Quilt-Aid Technology,
Johnson & Johnson) following inoculation with a load of approximately 2.5 × 10⁴ or 2.5 × 10⁶ colony
forming units (cfu) of the bacterium Escherichia coli and incubation at 37° C. for 24 hours.
Swatches were either washed or unwashed following application of the FSL construct.

| | unwashed | | | | washed | | | |
|---|---|---|---|---|---|---|---|---|
| Treatment | 1.99 × 10⁶ | 1.99 × 10⁶ | 1.99 × 10⁴ | 1.99 × 10⁴ | 1.99 × 10⁶ | 1.99 × 10⁶ | 1.99 × 10⁴ | 1.99 × 10⁴ |
| FSL-Se alone | Growth | Growth | Growth | Growth | Growth | Growth | Growth | Growth |
| FSL-Se with crystal | No growth | No growth | No growth | No growth | Inhibition | Inhibition | Inhibition | Inhibition |
| violet | | | | | | | | |
| FSL-Se with crystal | No growth | No growth | No growth | No growth | Inhibition | Inhibition | Inhibition | Inhibition |
| violet and AgNPs | | | | | | | | |
| FSL-spm | Growth | Growth | Growth | Growth | Growth | Growth | Growth | Growth |
| FSL-spm with crystal | Growth | Growth | Growth | Growth | Growth | Growth | Growth | Growth |
| violet | | | | | | | | |
| FSL-spm with crystal | Growth | Growth | No growth | No growth | Growth | Growth | Growth | Growth |
| violet and AgNPs | | | | | | | | |
| FSL-HA alone | Growth | Growth | Growth | Growth | Growth | Growth | Growth | Growth |
| FSL-HA with crystal | Growth | Growth | Growth | Growth | Growth | Growth | Growth | Growth |
| violet | | | | | | | | |
| FSL-HA with crystal | Growth | Growth | Growth | Growth | Growth | Growth | Growth | Growth |
| violet and AgNPs | | | | | | | | |
| FSL-0 alone | Growth | Growth | Growth | Growth | Growth | Growth | Growth | Growth |
| FSL-0 with crystal | No growth | No growth | No growth | No growth | Inhibition | Inhibition | Inhibition | Inhibition |
| violet | | | | | | | | |
| FSL-0 with crystal | No growth | No growth | No growth | No growth | Growth | Growth | No growth | No growth |
| violet and AgNPs | | | | | | | | |
| FSL-biotin alone | Growth | Growth | Growth | Growth | Growth | Growth | Growth | Growth |
| FSL-biotin with crystal | No growth | No growth | No growth | No growth | Inhibition | Inhibition | Inhibition | Inhibition |
| violet | | | | | | | | |
| FSL-biotin with crystal | No growth | No growth | No growth | No growth | Inhibition | Inhibition | Inhibition | Inhibition |
| violet and AgNPs | | | | | | | | |
| Crystal violet alone | Growth | Growth | Growth | Growth | Growth | Growth | Growth | Growth |
| Crystal violet and | Growth | Growth | Growth | Growth | Growth | Growth | Growth | Growth |
| AgNPs | | | | | | | | |
| AgNPs alone | Growth | Growth | Growth | Growth | Growth | Growth | Growth | Growth |
| Ag BAND AID | Inhibition | Inhibition | No growth | No growth | N.D. | N.D. | N.D. | N.D. |
| Positive control | Growth | Growth | Growth | Growth | Growth | Growth | Growth | Growth |
| (no treatment) | | | | | | | | |
| Negative control | No growth | No growth | No growth | No growth | No growth | No growth | No growth | No growth |
| (no treatment) | | | | | | | | |

N.D. (not determined).

TABLE 7
Observations of growth (growth), inhibition of growth (inhibition) or prevention of growth (no growth) on
duplicate treated square swatches (5 × 5 mm) of adhesive bandages (BAND-AID ™, Quilt-Aid Technology,
Johnson & Johnson) following inoculation with a load of approximately 2.5 × 10⁴ or 2.5 × 10⁶ colony
forming units (cfu) of the bacterium Staphylococcus epidermis and incubation at 37° C. for 24 hours.
Swatches were either washed or unwashed following application of the FSL construct.

| | unwashed | | | | washed | | | |
|---|---|---|---|---|---|---|---|---|
| Treatment | 2.11 × 10⁶ | 2.11 × 10⁶ | 2.11 × 10⁴ | 2.11 × 10⁴ | 2.11 × 10⁶ | 2.11 × 10⁶ | 2.11 × 10⁴ | 2.11 × 10⁴ |
| FSL-Se alone | Growth | Growth | Growth | Growth | Growth | Growth | Growth | Growth |
| FSL-Se with crystal | No growth | No growth | No growth | No growth | Inhibition | Inhibition | No growth | No growth |
| violet | | | | | | | | |
| FSL-Se with crystal | No growth | No growth | No growth | No growth | Inhibition | Inhibition | No growth | No growth |
| violet and AgNPs | | | | | | | | |
| FSL-spm | Growth | Growth | Growth | Growth | Inhibition | Inhibition | Inhibition | Inhibition |
| FSL-spm with crystal | Growth | Growth | Growth | Growth | Inhibition | Inhibition | Inhibition | Inhibition |
| violet | | | | | | | | |
| FSL-spm with crystal | Growth | Growth | Growth | Growth | Growth | Growth | Inhibition | Inhibition |
| violet and AgNPs | | | | | | | | |
| FSL-HA alone | Growth | Growth | Growth | Growth | Growth | Growth | Inhibition | Inhibition |
| FSL-HA with crystal | Growth | Growth | Growth | Growth | Growth | Growth | Inhibition | Inhibition |
| violet | | | | | | | | |
| FSL-HA with crystal | Growth | Growth | Growth | Growth | Growth | Growth | Inhibition | Inhibition |
| violet and AgNPs | | | | | | | | |
| FSL-0 alone | Growth | Growth | Growth | Growth | Growth | Growth | Growth | Growth |
| FSL-0 with crystal | No growth | No growth | No growth | No growth | Inhibition | Inhibition | Inhibition | Inhibition |
| violet | | | | | | | | |
| FSL-0 with crystal | No growth | No growth | No growth | No growth | Growth | Growth | No growth | No growth |
| violet and AgNPs | | | | | | | | |
| FSL-biotin alone | Growth | Growth | Inhibition | Inhibition | Growth | Growth | Inhibition | Inhibition |
| FSL-biotin with crystal | No growth | No growth | No growth | No growth | Inhibition | Inhibition | Inhibition | Inhibition |
| violet | | | | | | | | |
| FSL-biotin with crystal | No growth | No growth | No growth | No growth | Inhibition | Inhibition | Inhibition | Inhibition |
| violet and AgNPs | | | | | | | | |
| Crystal violet alone | Growth | Growth | Inhibition | Inhibition | Growth | Growth | Inhibition | Inhibition |
| Crystal violet and | Growth | Growth | Inhibition | Inhibition | Growth | Growth | Inhibition | Inhibition |
| AgNPs | | | | | | | | |
| AgNPs alone | Growth | Growth | Inhibition | Inhibition | Growth | Growth | Inhibition | Inhibition |
| Ag BAND AID | No growth | No growth | No growth | No growth | N.D. | N.D. | N.D. | N.D. |
| Positive control | Growth | Growth | Inhibition | Inhibition | Growth | Growth | Growth | Growth |
| (no treatment) | | | | | | | | |
| Negative control | No growth | No growth | No growth | No growth | No growth | No growth | No growth | No growth |
| (no treatment) | | | | | | | | |

N.D. (not determined).

TABLE 8
Observations of growth (growth), inhibition of growth (inhibition) or prevention of growth (no growth) on
duplicate treated square swatches (5 × 5 mm) of adhesive bandages (BAND-AID ™, Quilt-Aid Technology,
Johnson & Johnson) following inoculation with a load of approximately 2.5 × 10⁴ or 2.5 × 10⁶ colony
forming units (cfu) of the bacterium Pseudomonas aeruginosa and incubation at 37° C. for 24 hours.
Swatches were either washed or unwashed following application of the FSL construct.

| | unwashed | | | | washed | | | |
|---|---|---|---|---|---|---|---|---|
| Treatment | 1.75 × 10⁶ | 1.75 x 10⁶ | 1.75 x 10⁴ | 1.75 x 10⁴ | 1.75 x 10⁶ | 1.75 x 10⁶ | 1.75 x 10⁴ | 1.75 x 10⁴ |
| FSL-Se alone | Growth | Growth | Growth | Growth | Growth | Growth | Growth | Growth |
| FSL-Se with crystal | Inhibition | Inhibition | Inhibition | Inhibition | Growth | Growth | Growth | Growth |
| violet | | | | | | | | |
| FSL-Se with crystal | No growth | No growth | No growth | No growth | Inhibition | Inhibition | Inhibition | Inhibition |
| violet and AgNPs | | | | | | | | |
| FSL-spm | Growth | Growth | Growth | Growth | Growth | Growth | Growth | Growth |
| FSL-spm with crystal | Growth | Growth | Growth | Growth | Growth | Growth | Growth | Growth |
| violet | | | | | | | | |
| FSL-spm with crystal | Growth | Growth | No growth | No growth | Growth | Growth | No growth | No growth |
| violet and AgNPs | | | | | | | | |
| FSL-HA alone | Growth | Growth | Growth | Growth | Growth | Growth | Inhibition | Inhibition |
| FSL-HA with crystal | Growth | Growth | Growth | Growth | Growth | Growth | Inhibition | Inhibition |
| violet | | | | | | | | |
| FSL-HA with crystal | Growth | Growth | Inhibition | Inhibition | Growth | Growth | Inhibition | Inhibition |
| violet and AgNPs | | | | | | | | |
| FSL-0 alone | Growth | Growth | Growth | Growth | Growth | Growth | Growth | Growth |
| FSL-0 with crystal | Inhibition | Inhibition | No growth | No growth | Growth | Growth | Growth | Growth |
| violet | | | | | | | | |
| FSL-0 with crystal | Inhibition | Inhibition | No growth | No growth | Growth | Growth | Growth | Growth |
| violet and AgNPs | | | | | | | | |
| FSL-biotin alone | Growth | Growth | Inhibition | Inhibition | Growth | Growth | Growth | Growth |
| FSL-biotin with crystal | Inhibition | Inhibition | Inhibition | Inhibition | Growth | Growth | Growth | Growth |
| violet | | | | | | | | |
| FSL-biotin with crystal | No growth | No growth | No growth | No growth | Inhibition | Inhibition | Inhibition | Inhibition |
| violet and AgNPs | | | | | | | | |
| Crystal violet alone | Growth | Growth | Growth | Growth | Growth | Growth | Growth | Growth |
| Crystal violet and | Growth | Growth | Growth | Growth | Growth | Growth | Growth | Growth |
| AgNPs | | | | | | | | |
| AgNPs alone | Growth | Growth | Growth | Growth | Growth | Growth | Growth | Growth |
| Ag BAND AID | Inhibition | Inhibition | No growth | No growth | N.D. | N.D. | N.D. | N.D. |
| Positive control | Growth | Growth | Growth | Growth | Growth | Growth | Growth | Growth |
| (no treatment) | | | | | | | | |
| Negative control | No growth | No growth | No growth | No growth | No growth | No growth | No growth | No growth |
| (no treatment) | | | | | | | | |

N.D. (not determined).

used to inoculate a plate of Columbia Sheep Blood Agar. The plate was incubated at a temperature of 37° C. for a period of time of 18 to 22 hours. Individual colonies were used to inoculate a volume of 10 mL of Mueller Hinton broth and incubated with shaking at a temperature of 37° C. for a period of time of 4 hours. The optical density of the log phase culture was adjusted to provide a cell density in the range of 10⁶ to 10⁷ per μL.

A stock solution of crystal violet at a concentration of 8 mM was prepared by dissolving a quantity of 5 mg of this chloride salt in a volume of 1.5 mL of sterile deionised water. A stock solution of silver nitrate (AgNO₃) at a concentration of 16 mM was prepared by dissolving a quantity of 5 mg of this nitrate salt in a volume of 1.8 mL of sterile deionised water and protected from light. Both stock solutions were stored refrigerated and 10-fold dilutions prepared from these stock solutions. Equal volumes of these 10-fold dilutions were mixed to provide a combined solution containing both crystal violet and silver nitrate (an “antimicrobial composition”).

Dispersions of the lipidated polyanionic molecules (L-A-M) designated FSL-0 and FSL-0 at a concentration of 0.13 mM were prepared. To a quantity of 0.5 mg of FSL-0 a volume of 2.0 mL of the combined solution was added. To a quantity of 0.5 mg of FSL-0 a volume of 1.85 mL of the combined solution was added.

Swatches (0.25 cm²) of fabric from BAND-AID™ adhesive bandages (QUILT-AID™ technology, Johnson & Johnson) were impregnated with a volume of 50 μL of either dispersion and then dried at a temperature of 80° C. The dried swatches were repeatedly (3 times) washed with a volume of 10 mL deionised water in a petri dish by agitating for 15 seconds and aspirating. The impregnated, washed swatches were again dried at a temperature of 80° C. for a period of time of 30 minutes before being transferred to individual wells of a sterile 96-well microplate.

A volume of 10 μL of a log phase culture (10⁶ to 10⁷ cells) of a bacterial isolate was dispensed into each well and the plate incubated for a period of time of 10 minutes at room temperature before the addition of a volume of 50 μL of deionised water and a volume of 150 μL of Mueller Hinton broth. The plate was incubated with shaking (200 rpm) at a temperature of 37° C. for a period of time of 22 hours before recovering the swatches from each of the wells using sterile forceps.

Approximately 10 replicate swatches were transferred to a volume of 10 mL phosphate buffered saline (PBS) and vortexed for 30 seconds. A volume of 50 μL of the solution from the vortexed mixture was serially diluted (ten, one hundred and one thousand-fold) in 0.1% peptone water (Sigma-Aldrich). A volume of 100 μL of the solution from the vortexed mixture and each of the serial dilutions was used to inoculate a plate of Columbia Sheep Blood Agar and colonies developing on each plate counted following incubation at a temperature of 37° C. for a period of time of 24 hours.

Antibacterial Surface Treatments—Stainless Steel

A concave stamped coupon of stainless steel (SS316) was placed in each well of a 24-well culture plate. A volume of 50 μL of a dispersion of a lipidated polyanionic molecules (L-A-M) was dispensed on to the surface of each of the coupons followed by drying at a temperature of 80° C. Each of the dried coupons was washed repeatedly (3 times) in situ with a volume of 1 mL of deionised water by agitating for 15 seconds and aspirating.

Following drying at a temperature of 80° C. for a period of time of 45 minutes a volume of 10 μL of a log phase culture (10⁶ to 10⁷ cells) of a bacterial isolate (Staphylococcus aureus, Staphylococcus epidermidis, Pseudomonas aeruginosa and Escherichia coli) was dispensed onto the surface of each of the cooled coupons. The inoculated coupons were incubated at room temperature for a period of time of 10 minutes before dispensing a volume of 1 mL of Mueller Hinton broth amended with resazurin (23 mL broth plus 2 mL 0.02% resazurin) into each well. The 24-well plate was then incubated with shaking (200 rpm) at a temperature of 37° C. for a period of time of 22 hours.

The coupons were removed from the incubated 24-well plate and washed by immersing in deionised water before air drying at room temperature overnight. The growth of bacteria on the surface of the washed and dried coupons was visualised by staining with crystal violet or scanning electron microscopy (SEM). For staining a volume of 100 μL of a 0.4% solution of crystal violet was dispensed onto the surface of the stamped coupon and incubated at room temperature for a period of time of 10 minutes. Images were taken of the dried surface following the repeated (6 times) washing with deionised water. For SEM, samples were fixed with a solution of 2.5% glutaraldehyde.

Effect of Serum

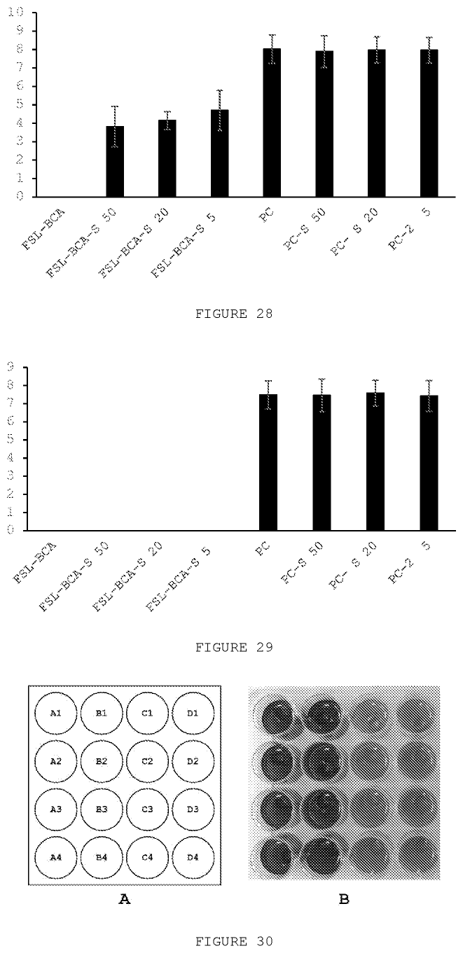

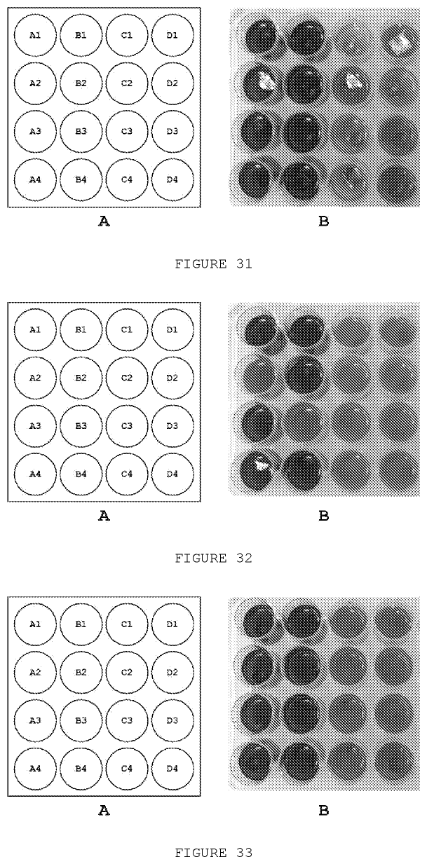

Swatches (0.25 cm²) of fabric from BAND-AID™ adhesive bandages (QUILT-AID™ technology, Johnson & Johnson) and concave stamped coupons of stainless steel (SS316) were treated as described above and the experiments repeated with the inclusion of a volume of 5, 20 or 50 μL of serum either applied directly to the surface (swatches) or added to the Mueller Hinton broth (concave stamped coupons of stainless steel) (FIGS. 26 to 34).

Although the invention has been described with reference to embodiments or examples it should be appreciated that variations and modifications may be made to these embodiments or examples without departing from the scope of the invention. Where known equivalents exist to specific elements, features or integers, such equivalents are incorporated as if specifically referred to in this specification. Variations and modifications to the embodiments or examples that include elements, features or integers disclosed in and selected from the referenced publications are within the scope of the invention unless specifically disclaimed. The advantages provided by the invention and discussed in the description may be provided in the alternative or in combination in these different embodiments of the invention.

### REFERENCED PUBLICATIONS

Adams (1967) The antibacterial action of crystal violet Journal of Pharmacy and Pharmacology 19(12), 821-826.
Bovin et al (2016) Antimicrobial surface treatment International application no. PCT/NZ2015/050181 [publ. no. WO 2016/072863 A1].
Docampo and Moreno (1990) The metabolism and mode of action of gentian violet Drug Metabolism Reviews 22(2-3), 161-178.
Resazurin-based 96-well plate microdilution method for the determination of minimum inhibitory concentration of biosurfactants Biotechnology Letters 38(6), 1015-1019.
Mann and Markham (1998) A new method for determining the minimum inhibitory concentration of essential oils Journal of Applied Microbiology 84(4), 538-544.
Montoro et al (2005) Comparative evaluation of the nitrate reduction assay, the MTT test, and the resazurin microtitre assay for drug susceptibility testing of clinical isolates of Mycobacterium tuberculosis Journal of Antimicrobial Chemotherapy 55(4), 500-505.
Pettit et al (2005) Microplate alamar blue assay for Staphylococcus epidermis biofilm susceptibility testing Antimicrobial Agents and Chemotherapy 49(7), 2612-2617.
Sarker et al (2007) Microtitre plate-based antibacterial assay incorporating resazurin as an indicator of cell growth, and its application in the in vitro antibacterial screening of phytochemicals Methods 42(4), 321-324.

1. A method of treating a surface comprising the steps of:
(a) contacting the surface with a lipidated polyanionic molecule dispersed in a solution of one or more water-dispersible antimicrobial agents; and then
(b) drying the surface,
where the antimicrobial activity of the one or more water-dispersible antimicrobial agents is retained at the surface when the surface is contacted with an aqueous vehicle.

2. The method of claim 1 where the one or more water-dispersible antimicrobial agents are selected from the group consisting of: silver and salts of hexamethylpararosaniline.

3. The method of claim 2 where the silver is in the form of silver nitrate (AgNO₃).

4. The method of claim 2 or 3 where the salt of hexamethylpararosaniline is hexamethylpararosaniline chloride (crystal violet).

5. The method of claim 1 where the surface is selected from the group consisting of ceramics, metals and polymers.

6. The method of claim 5 where the surface is selected from the group consisting of: the fabric of an adhesive bandage and the stainless steel of a dental or medical implant.

7. The method of claim 1 where the aqueous vehicle is water.

8. The method of claim 1 where the lipidated polyanionic molecule is of the structure L-A-M where L is a lipid, A is an optional linker and M is the polyanionic molecule.

9. The method of claim 8 where L is a lipid selected from the group consisting of: monoacyl-, monoalkyl-, diacyl- and dialkyl-lipids.

10. The method of claim 9 where the polyanionic molecule is a polycarboxylic molecule.

11. The method of claim 10 where the lipidated polyanionic molecule is a lipidated polycarboxylic molecule of the structure: 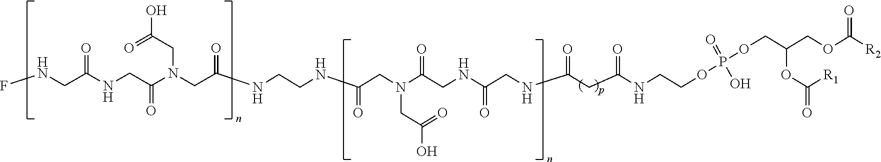
where F is H or selenocyanate, n is the integer 1, 2 or 4, p is the integer 3, 4 or 5 and R₁ and R₂ are independently selected from the group consisting of: saturated and mono-unsaturated C₁₆₋₂₀-acyl substituents.

12. A composition consisting essentially of a water-soluble salt of hexamethylpararosaniline, a water-soluble salt of silver, a lipidated polyanionic molecule and water.

13. The composition of claim 12 where the lipidated polyanionic molecule is a lipidated polycarboxylic molecule of the structure: 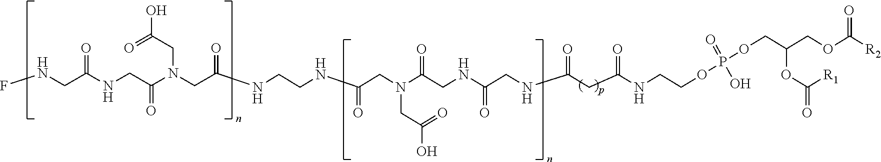
where F is H, n is the integer 1, 2 or 4, p is the integer 3, 4 or 5 and R₁ and R₂ are independently selected from the group consisting of: saturated and mono-unsaturated O₁₆₋₂₀-acyl substituents.

14. A fabric or dental or surgical implant having a surface treated according to the method of claim 1.

15. (canceled)
